(19) United States
(12) Patent Application Publication
(10) Pub. No.: US 2024/0082321 A1
(43) Pub. Date: Mar. 14, 2024

(54) STIMULATION OF THE GROWTH OF GUT BIFIDOBACTERIA

(71) Applicant: Adare Pharmaceuticals SAS, Houdan (FR)

(72) Inventors: Alicja WARDA, Cork (IE); Colin HILL, Cork (IE); Stephen PERRETT, Princeton, NJ (US)

(21) Appl. No.: 17/922,636

(22) PCT Filed: Apr. 30, 2021
(86) PCT No.: PCT/EP2021/061392

§ 371 Date: Nov. 1, 2022

Related U.S. Application Data
(60) Provisional application No. 63/018,908, filed on May 1, 2020.

(51) Int. Cl. A61K 35/747

(52) U.S. Cl. CPC A61K 35/747

(57) ABSTRACT

The present disclosure provides compositions for stimulating the growth of gut bifidobacteria, thereby maintaining/improving gut health. Of particular interest are compositions comprising cells of Lactobacillus fermentum, including the product LACTEOL®. Examples of specific conditions that may be treated which the compositions include antibiotic-associated diarrhea, dysbiosis, irritable bowel syndrome (IBS) and inflammatory bowel disease (IBD).

### CROSS-REFERENCE TO RELATED APPLICATIONS

This application is a U.S. National Stage Application under 35 U.S.C. § 371 of International Application No. PCT/EP2021/061392, filed Apr. 30, 2021, which claims priority to U.S. Provisional Application No. 63/018,908, filed May 1, 2020, the contents of each of which are incorporated herein by reference in their entireties.

### REFERENCE TO SEQUENCE LISTING

The instant application includes a Sequence Listing, which has been submitted electronically in a computer readable .txt format, and which is incorporated herein by reference in its entirety. The submitted .txt file, created on Apr. 19, 2023, is named: ADAR 164_01US_SubSeqList_ST25.txt, and has a size of approximately 1,554 bytes.

### BACKGROUND OF THE INVENTION

The present disclosure relates to agents capable of stimulating the growth of bifidobacteria in mammalian (e.g. human) gut.

Gut microbiota composition can play an important role in host health status. In particular, microbiota disruption has been linked with diarrhea, irritable bowel syndrome (IBS), obesity, allergies and behavioral and developmental disorders, including autism. Strategies designed to influence microbiota composition include the ingestion of probiotics, prebiotics, and synbiotics (a combination of probiotic bacteria and prebiotic stimulating proliferation of this and other bacteria). More drastic, but less predictable, approaches to microbiota modulation include supplementation of antimicrobials (such as antibiotics or bacteriocins) or fecal microbiota transfer (FMT). Altered levels of microbial metabolites have also been associated with conditions such as depression, colorectal cancer, cardiovascular disease, obesity and type 2 diabetes. Therefore, the role of microbially-derived molecules such as neurotransmitters, short-chain fatty acids (SCFAs), indoles, bile acids, choline metabolites, lactate and vitamins play an important role in health and well-being.

SCFAs are produced during the microbial fermentation of non-digestible dietary carbohydrates. SCFA production can contribute directly to host energy metabolism with acetate and propionate being absorbed and metabolized by the liver and peripheral organs, while butyrate is mainly utilized by the colonic epithelium, and can be used by certain bacteria as an energy source. Additionally, SCFAs have a beneficial effect on the host physiology by modulation of cell differentiation, anti-carcinogenic and anti-inflammatory effects or by enhancement of satiety and suppression of appetite. Production of propionate and acetate by bifidobacteria has been suggested as one reason for their beneficial effects on host health.

Bifidobacteria are anaerobic, Gram-positive bacteria often found in the human gastrointestinal tract. In healthy adults between 4.4% and 17.9% of the total fecal microbiota are bifidobacteria. Generally, higher levels of bifidobacteria have been associated with beneficial effects, including decreased levels of endotoxins in the gut, decreased intestinal permeability, decreased rates for bacterial translocation and metabolic improvements. At the same time, decreased numbers of bifidobacteria have been associated with diverse disorders including antibiotic-associated diarrhea, IBS, inflammatory bowel disease (IBD), obesity, allergies and regressive autism. Therefore, stimulation of Bifidobacterium is a valid strategy to prevent and/or reduce the extent of many disorders and improve quality of life. Stimulation of intrinsic Bifidobacterium spp. is of particular interest.

Lactobacillus is a genus of gram-positive, facultative anaerobic or microaerophilic, rod-shaped, non-spore-forming bacteria. They are a major part of the lactic acid bacteria group (i.e. they convert sugars to lactic acid). In humans, they constitute a significant component of the microbiota at a number of body sites. Lactobacillus currently contains over 180 species and encompasses a wide variety of organisms. For the purpose of the present disclosure, references to Lactobacillus include Lactobacillus fermentum, which was recently renamed as Limosilactobacillus fermentum. Thus, Lactobacillus fermentum and Limosilactobacillus fermentum are used interchangeably in this disclosure.

We have now surprisingly found that compositions comprising cells (e.g. dead cells) of strains of Lactobacillus and/or culture medium in which such cells have been grown, together with supernatant (i.e. cell free supernatant) and cell fractions thereof, are capable of stimulating the growth of bifidobacteria in mammalian (e.g. human) gut. These compositions and fractions are particularly useful for maintaining a healthy mammalian (e.g. human) gut, and for the treatment of disorders which may be aided by increased amounts of bifidobacteria in the gut, including antibiotic-associated diarrhea, dysbiosis, irritable bowel syndrome (IBS) and inflammatory bowel disease (IBD). A report summarizing aspects of the present disclosure may be found in Applied and Environmental Microbiology, doi: 10.1128/AEM.02459-20 (Warda et al.), posted online Feb. 12, 2021 (http://aem.asm.org). This article and its specific contents are incorporated herein by reference.

As used herein, “culture medium” is preferably MRS broth (i.e. traditional MRS product without the agar component) which includes material resulting from the growth of cells of one or more strains of Lactobacillus.

One particular product of the present disclosure capable of stimulating the growth of bifidobacteria in mammalian (e.g. human) gut is LACTEOL®. LACTEOL® is sold as a symptomatic treatment for diarrhea in adults and children supplemental to rehydration and/or dietary measures. However, it has not previously been reported to stimulating the growth of bifidobacteria in mammalian (e.g. human) gut.

Another particular product of the present disclosure capable of stimulating the growth of bifidobacteria in mammalian (e.g. human) gut is low lactose LACTEOL® (hereinafter referred to as LLL). LLL is a product of the manufacture of LACTEOL® prior to the addition of lactose to form the finished LACTEOL® product as sold. LLL contains less than 10% w/w lactose in LACTEOL®.

Another particular product of the present disclosure capable of stimulating the growth of bifidobacteria in mammalian (e.g. human) gut is a component of LACTEOL®, namely Lactobacillus fermentum.

Also of particular interest are fractions of the supernatant and cells (e.g. dead cells) of LACTEOL®, LLL and Lactobacillus fermentum, including a fraction of LACTEOL® supernatant designated Fraction 52, capable of stimulating the growth of bifidobacteria in mammalian (e.g. human) gut.

The present disclosure further relates to one or more compounds comprised within Fraction 52 responsible for stimulating the growth of bifidobacteria in mammalian (e.g. human) gut.

The active component in LACTEOL® is derived from a culture solution containing heat-killed cells of Lactobacillus LB strain (a combination of Lactobacillus fermentum, Lactobacillus delbrueckii) and fermented culture medium. LACTEOL®, along with other active products of this disclosure comprising dead Lactobacillus cells, have a number of potential advantages over products containing live organisms, such as probiotics, including consistency of composition and effect, ease of storage, no risk of infection in vulnerable patients, no translocation of bacterial-virulence or antibiotic-resistance cassettes, and the product retains activity when used in conjunction with antibiotics or anti-fungal agents.

### SUMMARY OF THE INVENTION

Throughout this document the terms “treatment” and “treating” are intended to also cover the preventative and protective uses of a composition of the present disclosure against a stated condition or disorder.

One aspect of the present disclosure provides a composition comprising cells (e.g. dead cells) of Lactobacillus fermentum for use in stimulating the growth of bifidobacteria in mammalian (e.g. human) gut.

Another aspect of the present disclosure provides a composition comprising cells (e.g. dead cells) of Lactobacillus fermentum and Lactobacillus delbrueckii for use in stimulating the growth of bifidobacteria in mammalian (e.g. human) gut.

A further aspect of the present disclosure provides a composition comprising the culture medium in which cells of Lactobacillus fermentum or cells of Lactobacillus fermentum and Lactobacillus delbrueckii were grown for use in stimulating the growth of bifidobacteria in mammalian (e.g. human) gut.

Another aspect of the present disclosure provides a composition comprising cells (e.g. dead cells) of Lactobacillus fermentum together with the culture medium in which cells of Lactobacillus fermentum were grown for use in stimulating the growth of bifidobacteria in mammalian (e.g. human) gut.

Yet another aspect of the present disclosure provides a composition comprising cells (e.g. dead cells) of Lactobacillus fermentum and Lactobacillus delbrueckii together with the culture medium in which cells of Lactobacillus fermentum and Lactobacillus delbrueckii were grown for use in stimulating the growth of bifidobacteria in mammalian (e.g. human) gut.

A further aspect of the present disclosure provides LACTEOL® for use in stimulating the growth of bifidobacteria in mammalian (e.g. human) gut.

Another aspect of the present disclosure provides LLL for use in stimulating the growth of bifidobacteria in mammalian (e.g. human) gut.

Yet another aspect of the present disclosure provides a supernatant fraction or cell fraction of LACTEOL® or LLL for use in stimulating the growth of bifidobacteria in mammalian (e.g. human) gut.

A particular aspect of the present disclosure provides a supernatant fraction of LACTEOL® or LLL for use in stimulating the growth of bifidobacteria in mammalian (e.g. human) gut.

A further particular aspect of the present disclosure provides Fraction 52 (as defined herein) for use in stimulating the growth of bifidobacteria in mammalian (e.g. human) gut.

Another particular aspect of the present disclosure provides one or more compounds within Fraction 52 for use in stimulating the growth of bifidobacteria in mammalian (e.g. human) gut.

One aspect of the present disclosure provides a method of improving gut health in an animal (including human) patient, comprising administering to the patient an effective amount of cells (e.g. dead cells) of Lactobacillus fermentum to increase the amount of bifidobacteria in mammalian (e.g. human) gut.

A further aspect of the present disclosure provides a method of improving gut health in an animal (including human) patient, comprising administering to the patient an effective amount of cells (e.g. dead cells) of Lactobacillus fermentum and cells (e.g. dead cells) of Lactobacillus delbrueckii, including cells (e.g. dead cells) of Lactobacillus LB to increase the amount of bifidobacteria in mammalian (e.g. human) gut.

Another aspect of the present disclosure provides a method of improving gut health in an animal (including human) patient, comprising administering to the patient an effective amount of the culture medium in which cells of Lactobacillus fermentum or cells of Lactobacillus fermentum and Lactobacillus delbrueckii were grown to increase the amount of bifidobacteria in mammalian (e.g. human) gut.

A further aspect of the present disclosure provides a method of improving gut health in an animal (including human) patient, comprising administering to the patient an effective amount of cells (e.g. dead cells) of Lactobacillus fermentum together with the culture medium in which cells of Lactobacillus fermentum were grown to increase the amount of bifidobacteria in mammalian (e.g. human) gut.

Yet another aspect of the present disclosure provides a method of improving gut health in an animal (including human) patient, comprising administering to the patient an effective amount of cells (e.g. dead cells) of Lactobacillus fermentum and Lactobacillus delbrueckii together with the culture medium in which cells of Lactobacillus fermentum and Lactobacillus delbrueckii were grown to increase the amount of bifidobacteria in mammalian (e.g. human) gut.

Another aspect of the present disclosure provides a method of improving gut health in an animal (including human) patient, comprising administering to the patient an effective amount of LACTEOL® to increase the amount of bifidobacteria in mammalian (e.g. human) gut.

A further aspect of the present disclosure provides a method of improving gut health in an animal (including human) patient, comprising administering to the patient an effective amount of LLL to increase the amount of bifidobacteria in mammalian (e.g. human) gut.

Yet another aspect of the present disclosure provides a method of improving gut health in an animal (including human) patient, comprising administering to the patient an effective amount of a supernatant fraction or cell fraction of LACTEOL® or LLL to increase the amount of bifidobacteria in mammalian (e.g. human) gut.

A particular aspect of the present disclosure provides a method of improving gut health in an animal (including human) patient, comprising administering to the patient an effective amount of a supernatant fraction of LACTEOL® or LLL to increase the amount of bifidobacteria in mammalian (e.g. human) gut.

A further aspect of the present disclosure provides a method of improving gut health in an animal (including human) patient, comprising administering to the patient an effective amount of Fraction 52 to increase the amount of bifidobacteria in mammalian (e.g. human) gut.

Another aspect of the present disclosure provides a method of improving gut health in an animal (including human) patient, comprising administering to the patient an effective amount of one or more compounds of Fraction 52 which exhibit an ability to increase the amount of bifidobacteria in mammalian (e.g. human) gut.

As used herein, the terms “improve gut health” and “improving gut health” include: (1) the prophylactic use of products of this disclosure to stimulate the growth of bifidobacteria in mammalian (e.g. human) gut to prevent or mitigate the occurrence of a gut disorder such as dysbiosis and (2) the use of products of this disclosure to stimulate the growth of bifidobacteria in mammalian (e.g. human) gut to treat disorders of the gut and GI tract, including antibiotic-associated diarrhea, dysbiosis, irritable bowel syndrome (IBS) and inflammatory bowel disease (IBD).

One aspect of the present disclosure provides a pharmaceutical composition comprising cells (e.g. dead cells) of Lactobacillus fermentum together with one or more pharmaceutically acceptable carriers or excipients for use in improving gut health in an animal (including human) patient by increasing the amount of bifidobacteria in mammalian (e.g. human) gut.

A further aspect of the present disclosure provides a pharmaceutical composition comprising cells (e.g. dead cells) of Lactobacillus fermentum and cells (e.g. dead cells) of Lactobacillus delbrueckii, including cells (e.g. dead cells) of Lactobacillus LB, together with one or more pharmaceutically acceptable carriers or excipients for use in improving gut health in an animal (including human) patient by increasing the amount of bifidobacteria in mammalian (e.g. human) gut.

Another aspect of the present disclosure provides a pharmaceutical composition comprising the culture medium in which cells of Lactobacillus fermentum or cells of Lactobacillus fermentum and Lactobacillus delbrueckii were grown, together with one or more pharmaceutically acceptable carriers or excipients for use in improving gut health in an animal (including human) patient by increasing the amount of bifidobacteria in mammalian (e.g. human) gut.

A further aspect of the present disclosure provides a pharmaceutical composition comprising cells (e.g. dead cells) of Lactobacillus fermentum together with the culture medium in which cells of Lactobacillus fermentum were grown, together with one or more pharmaceutically acceptable carriers or excipients for use in improving gut health in an animal (including human) patient by increasing the amount of bifidobacteria in mammalian (e.g. human) gut.

Yet another aspect of the present disclosure provides a pharmaceutical composition comprising cells (e.g. dead cells) of Lactobacillus fermentum and Lactobacillus delbrueckii together with the culture medium in which cells of Lactobacillus fermentum and Lactobacillus delbrueckii were grown, together with one or more pharmaceutically acceptable carriers or excipients for use in improving gut health in an animal (including human) patient by increasing the amount of bifidobacteria in mammalian (e.g. human) gut.

Another aspect of the present disclosure provides a pharmaceutical composition comprising an effective amount of LACTEOL® for use in improving gut health in an animal (including human) patient by increasing the amount of bifidobacteria in mammalian (e.g. human) gut.

Yet another aspect of the present disclosure provides a pharmaceutical composition comprising an effective amount of LLL, together with one or more pharmaceutically acceptable carriers or excipients, for use in improving gut health in an animal (including human) patient by increasing the amount of bifidobacteria in mammalian (e.g. human) gut.

Another aspect of the present disclosure provides a pharmaceutical composition comprising an effective amount of a supernatant fraction or cell fraction of LACTEOL® or LLL, together with one or more pharmaceutically acceptable carriers or excipients, for use in improving gut health in an animal (including human) patient by increasing the amount of bifidobacteria in mammalian (e.g. human) gut.

A particular aspect of the present disclosure provides a pharmaceutical composition comprising an effective amount of a supernatant fraction of LACTEOL® or LLL, together with one or more pharmaceutically acceptable carriers or excipients, for use in improving gut health in an animal (including human) patient by increasing the amount of bifidobacteria in mammalian (e.g. human) gut.

A further aspect of the present disclosure provides a pharmaceutical composition comprising an effective amount of Fraction 52, together with one or more pharmaceutically acceptable carriers or excipients, for use in improving gut health in an animal (including human) patient by increasing the amount of bifidobacteria in mammalian (e.g. human) gut.

Another aspect of the present disclosure provides a pharmaceutical composition comprising an effective amount of one or more compounds of Fraction 52, together with one or more pharmaceutically acceptable carriers or excipients, for use in improving gut health in an animal (including human) patient by increasing the amount of bifidobacteria in mammalian (e.g. human) gut.

### DESCRIPTION OF THE DRAWINGS

FIG. 1 shows the Bifidobacterium load and equivalent of total counts before and after 24-hour fecal fermentation in vessels supplemented with water, lactic acid, lactose or LACTEOL®.

FIG. 2A shows the effect of LACTEOL® on the growth of a range of infant- and adult-associated Bifidobacterium strains in 10× diluted media. FIG. 2B shows the effect of LACTEOL® components [supernatant, cells, and Low Lactose LACTEOL® (LLL)] on growth in 10× diluted media. FIG. 2C shows the effect of LACTEOL® dose on growth in 10× diluted media.

FIG. 2D and FIG. 2E show the effect of enzymatically or physically treated LACTEOL® on growth in 10× diluted media. FIG. 2F shows the effect of LACTEOL®-like preparations on the growth in 10× diluted media. FIG. 2G shows the effect of LLL and Lb. fermentum components [supernatant, cells, and dialyzed] on the growth in 15× diluted media.

FIGS. 3A and 3B show the effect of probiotics on 24 h growth of B. longum subsp. infantis ATCC 15697 (B1) in 15× diluted media.

FIGS. 4A, 4B and 4C show SPE C18 purification of LACTEOL®, Lb. fermentum, Lb. fermentum APC249, and LLL on the 24 h growth of B. longum subsp. infantis ATCC 15697.

FIG. 5 shows the effect of C18 purification of ammonium precipitation fractions on the Bifidobacterium growth in 10× diluted media.

FIG. 6 shows the UV absorption chromatograms of LACTEOL® and its fraction passed through a size exclusion column.

FIG. 7 shows the Bifidobacterium growth in response to HPLC fractions of C-18 purified ½ strength LACTEOL® (15× diluted media).

FIG. 8 shows the MALDI TOF Mass Spectrometry results for Fraction 52 and surrounding fractions of C18 purified LACTEOL®.

FIG. 9 shows the concentration effect of LACTEOL®-like preparations on 24 h growth of Bifidobacterium (15× diluted media). FIG. 9A shows the effect of the supernatant of Lb. fermentum strains at full and half of the weight equivalent of LACTEOL®. FIG. 9B shows the effect of combination of cells and supernatant at full and half of the weight equivalent of LACTEOL®.

FIG. 10 shows the effect of concentrated MRS preparations (cMRS; 3.4 g/10 ml water) on the growth of Bifidobacterium in 10× diluted media (FIG. 10A) and 15× diluted media (FIG. 10B).

### DETAILED DESCRIPTION

The present disclosure relates to microbiological compositions which stimulate the growth of bifidobacteria in the mammalian (e.g. human) gut, thereby helping to maintain and improve gut health.

Suitable compositions of the disclosure comprise, in one aspect, cells (e.g. dead cells) of Lactobacillus fermentum, or a mixture of cells (e.g. dead cells) of Lactobacillus fermentum and Lactobacillus delbrueckii, including Lactobacillus LB. A particular composition of the disclosure comprises LACTEOL®.

In another aspect, suitable compositions of the disclosure comprise components of LACTEOL®, LLL or Lactobacillus fermentum.

In a particular aspect, suitable compositions include the supernatant and/or cells of LACTEOL®, LLL or Lactobacillus fermentum.

A particular composition of the disclosure comprises fractions of the supernatant of LACTEOL® or LLL. One such fraction of particular interest is Fraction 52.

In another aspect, suitable compositions of the disclosure comprise one or more compounds within Fraction 52 responsible for stimulating the growth of beneficial bifidobacteria in mammalian (e.g. human) gut.

In a further aspect, suitable compositions of the disclosure comprise the culture medium in which cells of Lactobacillus fermentum or cells (e.g. dead cells) of Lactobacillus fermentum and Lactobacillus delbrueckii are grown.

In another aspect, suitable compositions of the disclosure comprise cells (e.g. dead cells) of Lactobacillus fermentum together with the culture medium in which cells of Lactobacillus fermentum are grown.

In yet another aspect, suitable compositions of the disclosure comprise cells (e.g. dead cells) of Lactobacillus fermentum and Lactobacillus delbrueckii together with the culture medium in which cells of Lactobacillus fermentum and Lactobacillus delbrueckii are grown.

Biological deposits referred to herein were deposited at the Collection Nationale de Cultures de Microorganismes (CNCM), located at Institut Pasteur, 25-28 rue du Docteur Roux, 75724 Paris Cedex 15. Lactobacillus LB in fermented culture medium is deposited at the CNCM with the reference code MA 65/4E (MA65/4E-1b: Lactobacillus fermentum, MA65/4E-2z: Lactobacillus delbrueckii). MA65/4E was deposited on 26 Aug. 1991. The two individual bacterial strains of Lactobacillus LB are recorded under the Budapest Treaty as Lactobacillus fermentum, CNCM 1-2998 (Ref: MA65/4E-1b; initial deposit date: 26 Aug. 1992; date of conversion to conform to the Budapest Treaty: 27 Mar. 2003) and Lactobacillus delbrueckii, CNCM 1-4831 (Ref: MA65/4E-2z; initial deposit date: 26 Aug. 1992; request to convert the initial deposit to conform to the Budapest Treaty: 20 Dec. 2013; conversion receipt issued 9 Jan. 2014).

Dead Lactobacillus LB cells may be obtained by heating the live cells in fermented culture medium at about 110° C. for about 1 hour. Dead cells of Lactobacillus fermentum, Lactobacillus delbrueckii or a mixture thereof may be obtained in a similar manner via a heat-killing process.

When used as a mixture, the weight ratio of Lactobacillus fermentum to Lactobacillus delbrueckii may be any suitable ratio from about 99:1 to about 1:99, e.g. about 9:1 to 1:9, including 9:1, 8:2, 7:3, 6:4, 5:5, 4:6, 3:7, 2:8, 1:9. The weight ratio of Lactobacillus fermentum to Lactobacillus delbrueckii may particularly be about 9:1.

LACTEOL® may be prepared by drying dead cells of Lactobacillus LB together with the fermented culture medium (e.g. by lyophilization, spray-drying or fluid-bed drying) prior to formulating into a suitable composition for use in the present invention. In a particular aspect, lactose may be added to the wet fermented product prior to drying. In another aspect, lactose may also be added after drying as part of the formulation step.

LACTEOL® contains a dried combination of heat-killed Lactobacillus fermentum and Lactobacillus delbrueckii in about a 9:1 ratio in culture medium.

Dead cells of Lactobacillus fermentum, Lactobacillus delbrueckii or a mixture thereof, including Lactobacillus LB, may also be used in a liquid form, with or without lactose, by omitting the drying step or reconstituting the dried product with a suitable liquid such as water.

Supernatant and cells of LACTEOL®, LLL or Lactobacillus fermentum may be prepared from solutions of LACTEOL®, LLL or Lactobacillus fermentum respectively by traditional separation techniques, such as centrifugation followed by separation of solid material from liquid (e.g. by filtration). Fractions of the supernatant of LACTEOL® or LLL may be obtained via size exclusion chromatography, such as size exclusion HPLC column chromatography. Fractions may be purified/concentrated using solvent-solvent extraction and by solid phase extraction column, e.g. C18, purification. Active compounds from within Fractions may be analyzed and characterized using LC-MS/MS (liquid chromatography, tandem mass spectrometry).

Fractions 52 is a fraction of the supernatant of LACTEOL® or LLC obtained by size exclusion HPLC and purified with multiple runs with C18; at MALDI TOF mass spectrometry Fraction 52 has a single peak at around 5200 m/z (such as at 5237.08 m/z).

In one aspect, cells (e.g. dead cells) of Lactobacillus fermentum, Lactobacillus delbrueckii or a mixture thereof, including Lactobacillus LB, are present in a composition of the present disclosure in a sufficient amount to achieve the desired effect. In one exemplary embodiment of the present disclosure, dead cells of the Lactobacillus LB strain are present in the proportion of about 1 billion or more cells/g, for example from about 10 to about 100 billion cells/g, including about 40 to about 80 billion cells/g (e.g. about 60 billion cells/g) in a composition of the present disclosure.

A composition of the present disclosure may be orally administered, and at a suitable dose, which will vary according to factors such as the subject's age, body weight and gender, the condition to be treated, and the duration of administration and the administration route. Ordinarily trained doctors or veterinarians can easily determine and prescribe an effective dose of a pharmaceutical composition of the present disclosure for the respective human or non-human animal patient. A pharmaceutical composition of the present disclosure in a suitable dosage form may be conveniently administered to the patient once or twice daily. In infants or younger children, based on a body weight ranging from 20 to 40 kg, approximately ½ of the adult dosage may be administered, and based on a body weight of less than 20 kg, approximately ¼ of the adult dosage may be administered.

A convenient unit dose of a composition of the present disclosure, e.g. in a standard pharmaceutical dosage form, such as a capsule, or a tablet, or a sachet, may be any effective dose up to about 2000 mg administered to an adult human patient once or twice daily.

A composition of the present disclosure may also be administered as a food or nutritional supplement or in a food, e.g. yoghurt. In this case very high doses up to about 100 g could be ingested.

A pharmaceutical composition of the present disclosure may be formulated using a pharmaceutically available carrier and/or excipient, and prepared in a unit capacity or contained in a high-dosage container according to a method that can be easily executed by one of ordinary skill in the art. Here, a dosage form may be a tablet, a capsule, a granule, powder, sachet containing powder, or liquids such as an aqueous medium-containing solution, a suspension, or an emulsion.

For example, in one aspect, to formulate a pharmaceutical composition as a capsule, dried (e.g. lyophilized) cells of Lactobacillus fermentum, or a mixture of cells of Lactobacillus fermentum and Lactobacillus delbrueckii, including Lactobacillus LB, (optionally together with fermented culture medium and/or lyophilization additives) may be mixed with one or more suitable, non-toxic pharmaceutically available inactive carriers and excipients. Examples include binding agents, lubricants, disintegrating agents, diluents, coloring agents and desiccants. Suitable binding agent may be, but is not limited to, natural sugar such as starch, gelatin, glucose, or beta-lactose, a natural or synthetic gum such as corn sweetener, acacia, Tragacanth, or sodium oleate, sodium stearate, magnesium stearate, sodium benzoate, sodium acetate, or sodium chloride. The disintegrating agent includes, but is not limited to, starch, methylcellulose, agar, bentonite, or xanthan gum. Suitable lubricants include talc and magnesium stearate. Suitable desiccants include silicic acid and suitable diluents include a lactose such as anhydrous lactose. Suitable lyophilization additives include lactose monohydrate and a metal carbonate such as calcium carbonate. The product mixture may be contained in any standard capsule casing such as in a gelatin capsule.

In another aspect, a pharmaceutical composition of the present disclosure, in the form of a powder for an oral suspension, may be prepared, for example, by mixing dried (e.g. lyophilized) cells of Lactobacillus fermentum, or cells of Lactobacillus fermentum and Lactobacillus delbrueckii (optionally together with fermented culture medium and/or lyophilization additives) with one or more suitable, non-toxic pharmaceutically available inactive carriers and excipients. Examples include diluents, flavoring agents, sweetening agents and desiccants. Suitable desiccants include silicic acid and suitable diluents include a lactose such as anhydrous lactose or sucrose, the latter may also act as a sweetening agent. Suitable lyophilization additives include lactose monohydrate and a metal carbonate such as calcium carbonate. The powder product may be contained in any standard sachet ready for mixing with a drinkable liquid.

A composition for oral administration may also be part of a liquid or solid food or nutritional product (e.g. nutritional supplement). Examples include a milk, yoghurt or yoghurt-style product, a cheese, an ice-cream, a cereal-based product, a milk-based powder, a nutritional formula, an infant formula, a nutritional formula, a dried oral grit or powder, a wet oral paste or jelly, a grit or powder for dry tube feeding or a fluid for wet tube feeding.

Furthermore, optional additional active ingredients may also be present for use with a composition of the present disclosure. Optional active ingredients include, for example, vitamins, antibiotics, probiotics or prebiotics. The additional active ingredient(s) and a composition of the present disclosure may be co-administered or administered separately (e.g. sequentially) as individual compositions. Alternatively, the active ingredient(s) may be incorporated into the same composition as the cells (e.g. dead cells) of Lactobacillus fermentum, or a mixture of cells (e.g. dead cells) of Lactobacillus fermentum and Lactobacillus delbrueckii, including Lactobacillus LB, optionally together with fermented culture medium and/or lyophilization additives.

The compositions of this disclosure and fractions thereof are particularly useful for maintaining a healthy mammalian (e.g. human) gut, and for the treatment of disorders which may be aided by increased amounts of bifidobacteria in the gut, including antibiotic-associated diarrhea, dysbiosis, irritable bowel syndrome (IBS) and inflammatory bowel disease (IBD).

The following are specific embodiments of the present disclosure:
Embodiment 1: A method of protecting a human or non-human animal subject against the development of a gut disorder, comprising administering to the subject an effective amount of cells of Lactobacillus fermentum to stimulate the growth of bifidobacteria in the human or non-human animal gut
Embodiment 2: A method of protecting a human or non-human animal subject against the development of a gut disorder, comprising administering to the subject effective amounts of cells of Lactobacillus fermentum and cells of Lactobacillus delbrueckii to stimulate the growth of bifidobacteria in the human or non-human animal gut Embodiment 3: A method according to Embodiment 1 or 2, wherein the cells of Lactobacillus fermentum and/or Lactobacillus delbrueckii are dead cells.
Embodiment 4: A method of protecting a human or non-human animal subject against the development of a gut disorder, comprising administering to the subject an effective amount of the product LACTEOL® to stimulate the growth of bifidobacteria in the human or non-human animal gut
Embodiment 5: A method of protecting a human or non-human animal subject against the development of a gut disorder, comprising administering to the subject an effective amount of the supernatant of the culture medium in which cells (e.g. dead cells) of Lactobacillus fermentum and cells (e.g. dead cells) of Lactobacillus delbrueckii have been grown to stimulate the growth of bifidobacteria in the human or non-human animal gut
Embodiment 6: A method of protecting a human or non-human animal subject against the development of a gut disorder, comprising administering to the subject an effective amount of Fraction 52 of the supernatant of Embodiment 5 to stimulate the growth of bifidobacteria in the human or non-human animal gut
Embodiment 7: A method according to any one of Embodiments 1-6, wherein the subject is a healthy human.
Embodiment 8: A method of treating a gut disorder in a human or non-human animal subject, comprising administering to the subject an effective amount of cells of Lactobacillus fermentum to stimulate the growth of bifidobacteria in the human or non-human animal gut Embodiment 9: A method of treating a gut disorder in a human or non-human animal subject, comprising administering to the subject effective amounts of cells of Lactobacillus fermentum and cells of Lactobacillus delbrueckii to stimulate the growth of bifidobacteria in the human or non-human animal gut Embodiment 10: A method according to Embodiment 8 or 9, wherein the cells of Lactobacillus fermentum and/or Lactobacillus delbrueckii are dead cells.
Embodiment 11: A method of treating a gut disorder in a human or non-human animal subject, comprising administering to the subject an effective amount of the product LACTEOL® to stimulate the growth of bifidobacteria in the human or non-human animal gut
Embodiment 12: A method of treating a gut disorder in a human or non-human animal subject, comprising administering to the subject an effective amount of the supernatant of the culture medium in which cells (e.g. dead cells) of Lactobacillus fermentum and cells (e.g. dead cells) of Lactobacillus delbrueckii have been grown to stimulate the growth of bifidobacteria in the human or non-human animal gut
Embodiment 13: A method of treating a gut disorder in a human or non-human animal subject, comprising administering to the subject an effective amount of Fraction 52 of the supernatant of
Embodiment 12 to stimulate the growth of bifidobacteria in the human or non-human animal gut, wherein Fraction 52 is a fraction of the supernatant of LACTEOL® or LLC obtained by size exclusion HPLC and purified with multiple runs with C18; at MALDI TOF mass spectrometry it has a single peak at around 5200 m/z (such as at 5237.08 m/).
Embodiment 14: A method according to any one of Embodiments 8-13, wherein the subject is a human.
Embodiment 15: A method according to any one of Embodiments 8-14, wherein the gut disorder is selected from antibiotic-associated diarrhea, dysbiosis, irritable bowel syndrome (IBS) and inflammatory bowel disease (IBD).
Embodiment 16: A composition comprising cells of Lactobacillus fermentum for use in maintaining and/or improving gut health of a human or non-human animal subject by stimulating the growth of bifidobacteria in the human or non-human animal gut.
Embodiment 17: A composition according to Embodiment 15, also comprising cells of Lactobacillus delbrueckii.
Embodiment 18: A composition comprising the culture medium in which cells of Lactobacillus fermentum have been grown for use in maintaining and/or improving gut health of a human or non-human animal subject by stimulating the growth of bifidobacteria in the human or non-human animal gut.
Embodiment 19: A composition comprising cells of Lactobacillus fermentum and the culture medium in which cells of Lactobacillus fermentum have been grown for use in maintaining and/or improving gut health of a human or non-human animal subject by stimulating the growth of bifidobacteria in the human or non-human animal gut.
Embodiment 20: A composition comprising cells of Lactobacillus fermentum and cells of Lactobacillus delbrueckii together with the culture medium in which cells of Lactobacillus fermentum and cells of Lactobacillus delbrueckii have been grown for use in maintaining and/or improving gut health of a human or non-human animal subject by stimulating the growth of bifidobacteria in the human or non-human animal gut.
Embodiment 21: A composition according to any one of Embodiments 16-20, wherein the cells of Lactobacillus fermentum and/or Lactobacillus delbrueckii are dead cells.
Embodiment 22: A composition comprising LACTEOL® for use in maintaining and/or improving gut health a human or non-human animal subject by stimulating the growth of bifidobacteria in the human or non-human animal gut.
Embodiment 23: A composition comprising the supernatant of the culture medium in which cells (e.g. dead cells) of Lactobacillus fermentum and cells (e.g. dead cells) of Lactobacillus delbrueckii have been grown for use in maintaining and/or improving gut health of a human or non-human animal subject by stimulating the growth of bifidobacteria in the human or non-human animal gut.
Embodiment 24: A composition comprising Fraction 52 of the supernatant of Embodiment 22 for use in maintaining and/or improving gut health of a human or non-human animal subject by stimulating the growth of bifidobacteria in the human or non-human animal gut.
Embodiment 25: A composition for use according to any one of Embodiments 16-24, wherein the subject is a healthy human.
Embodiment 26: A composition for use according to any one of Embodiments 16-24, for the treatment of a disorder of the gut.
Embodiment 27: A composition for use according to any one of Embodiments 16-24, for the treatment of a disorder of the gut in a human subject.
Embodiment 28: A composition for use according to Embodiment 26 or 27, wherein the gut disorder is selected from antibiotic-associated diarrhea, dysbiosis, irritable bowel syndrome (IBS) and inflammatory bowel disease (IBD).
Embodiment 29: A composition for use according to any one of Embodiments 16-28, wherein the composition is in the form of a pharmaceutical composition, a food supplement, or a nutritional supplement.
Embodiment 30: A composition for use according to Embodiment 29, wherein said food supplement or nutritional supplement is comprised within a food product selected from milk, yoghurt or yoghurt-style product, cheese, ice-cream, a cereal-based product, a milk-based powder, an infant formula, a nutritional formula, a dried oral grit or powder, a wet oral paste or jelly, a grit or powder for dry tube feeding or a fluid for wet tube feeding.

While the present disclosure has been described herein with reference to certain exemplary embodiments and specific Examples, it will be understood by those skilled in the art that modifications in form and details may be made therein without departing from the spirit and scope of the invention.

### EXAMPLES

### Example 1

### LACTEOL® Preparations

LACTEOL® powder was used for feeding fecal fermentation vessels.

### Fecal Fermentation—Preparation of Frozen Standardized Inoculum (FSI)

The frozen standardized inoculum was prepared in a manner similar to the method described by O'Donell M M et al. in Journal of Microbiological Methods. (2016) vol. 9, pages 9-16. Briefly, volunteers/donors (n=5) adhered to strict criteria: all donors were healthy adults and did not take antibiotics for the six months before donation. The fecal samples from donors were collected into plastic containers, placed in zip bags with generators of anaerobic condition (GENbox anaer, BioMerieux, France) and stored at 4° C. Within on average 9 h fecal samples were transferred into an anaerobic chamber (Don Whitley, West Yorkshire, UK) under an anoxic atmosphere (10% H₂, 0% 02, 0% N₂). The feces were pooled together into a large stomacher bag with a 70 μm filter insert (Sparks lab supplies, Ireland) in an anaerobic chamber. Four hundred millilitres of 50 mM phosphate buffer with 0.05% (w/v) 1-cysteine hydrochloride (Sigma Aldrich, Ireland) pH 6.8 (further referred as phosphate buffer) was added to the stomacher bag followed by manual sample homogenization. The filtered slurry was then centrifuged at 4000×g for 25 min in a Sorvall SLA-3000 centrifuge and resuspended in 400 ml phosphate buffer, again in an anaerobic cabinet. Next, second centrifuged (4000×g for 25 min) took place followed by resuspension in 400 ml phosphate buffer. The resulting fecal bacteria suspension was then supplemented with 200 ml of glycerol, aliquoted and frozen at −80° C. for 1-9 weeks until use (hereafter referred to as FSI). Except for centrifugation step all processing took place in the anaerobic cabinet. Before the use of FSI, the aliquots were thawed at 37° C. over 0.5-1 h before inoculation into fermentation vessels.

### Fecal Fermentation—Distal Colon Model

Starch-supplemented fecal medium was prepared as described by Fooks L J and Gibson G R in Anaerobe (2003) vol. 9(5), pages 231-42, with the final concentration in the fermentation vessel (total volume 200 ml) per liter: 2 g peptone, 2 g yeast extract, 0.76 g NaCl, 0.04 g K₂HPO₄, 0.04 g KH2PO₄, 0.007 g CaCl₂·2H₂O, 0.01 g MgSO₄·7H₂O, 2 g NaHCO₃, 2 ml Tween 80, 0.5 g L-cysteine-HCl, 0.5 g bile salts, 10 g soluble starch, 0.05 g hemin (dissolved in three drops of 1 M NaOH), and 10 μl Vitamin K1 (Sigma Aldrich). 180 ml of base media was supplemented with either 3.4 g/100 ml LACTEOL® (corresponding to 10 sachets/capsules of LACTEOL® at 340 mg in 100 ml), or equivalent amounts of lactic acid (30 mM), lactose (36 mM) or water as a control. When required water was added to bring volume to 187.5 ml. The medium was added to fermentation vessels of MultiFors system (Infors, UK), its pH was adjusted to 6.8, and each vessel was sparged with oxygen-free N₂ for at least 120 min to ensure anaerobic conditions were established. 12.5 ml FSI was used to inoculate each vessel. Fermentations were performed over 24 h at 37° C., maintained at constant pH 6.8 by the automatic addition of 1M NaOH or 1M HCl; sparged with oxygen-free N₂ and continuously stirred at 200 rpm. Samples were withdrawn from each of the vessels at TO, 1 h (T1), 2 h (T2), 3 h (T3), 4 h (T4), 5 h (T5), 6 h (T6), 22 (T22) and after 24 h (T24) of fermentation and stored at −80° C. until processing. Each of the conditions was tested at least in triplicate.

### DNA Isolation

DNA isolation from fecal fermentation samples was performed using QIAamp Fast DNA Stool Mini kit (Qiagen, Germany) according to the manufacturer's recommendations with minor modifications, increasing the volume of used bead-beated (FastPrep-24, MP Biomedicals, United States) solution to 600 μl and decreasing the final elution volume to 30 μl TAE. Assessment of DNA quantity and quality was performed by measurement of DNA concentration using Qubit dsDNA BR Assay Kit and running 5 μl sample on a gel for quality assessment.

### 16S Metagenomics—Microbiota Analysis

### DNA Amplification, Indexing, Normalization and Sequencing

Library preparation was performed as described by Warda A K et al. in Behavioural brain research (2018) vol. 362, pages 213-23. V3 and V4 region of 16S genes were amplified using Phusion Polymerase Master Mix and V3-V4 (Forward 5′-TCGTCGGCAGCGTCAGAT GTGTATAAGAGACAGCCTACGGGNGGCWGCAG-3′ (SEQ ID NO: 1); Reverse 5′-GTCTCGTGGGCTCGGAGATGTGTATAAGAGACAGGACTACHVGGGTATCTAATCC-3′ (SEQ ID NO: 2)) primers (98° C. 30 s; 25 cycles of 98° C. 10 s, 55° C. 15 s, 72° C. 20 s; 72° C. 5 min). Amplicons were checked for quality and quantity by Qubit dsDNA HS Assay Kit and running on gel, and cleaned using Ampure XP magnetic beads. 5 μl of the cleaned amplicon was used as template for Index PCR using Phusion Polymerase Master Mix and Nextera XT Index Kit (95° C. 30 s; 8 cycles of 95° C. 30 s, 55° C. 30 s, 72° C. 30 s; 72° C. 5 min). Indexed amplicons were cleaned using Ampure XP magnetic beads and checked for quality and quantity by Qubit dsDNA HS Assay Kit and running on gel. All samples were normalised in water to 4 nM, followed by pooling together 5 μl of each sample and sending for Illumina MiSeq sequencing to GTAC (Germany).

### 16S Data Analysis

The quality of the raw reads was visualized with FastQC v0.11.3. The reads were then imported into R v3.3.0 for analysis with the DADA2 package (v1.03) according to Callahan B J et al. in Nat Methods (2016) vol. 13(7), pages 581-3. Errors introduced during the sequencing process were corrected to generate Ribosomal Sequence Variants (RSVs). These were exported and further chimera filtered using both the de novo and reference-based chimera filtering implemented in USEARCH v8.1.1861 with the ChimeraSlayer gold database v20110519. The remaining RSVs were classified with mothur v1.38 [see Schloss P D et al. in Applied and environmental microbiology (2009) vol. 75(23), pages 7537-41] against the RDP database version 11.4, as well as classified with SPINGO to species level [see Allard G et al. in BMC bioinformatics (2015) vol, 16, page 324]. Only RSVs with a domain classification of Bacteria or Archaea were kept for further analysis. A phylogenetic tree of the RSV sequences rooted on the midpoint was generated with FastTree [see Price M N et al. in Molecular biology and evolution (2009) vol. 26(7), pages 1641-50]. Alpha diversity and beta diversity were generated using PhyloSeq v1.16.2, which also was used for a principle coordinates analysis as implemented in Ape v3.5. Differential abundance analysis was carried out with DESeq2 v1.12.4 [see Love M I et al. in Genome Biol. (2014) vol. 15(12), page 550] for RSV level and Wilcoxon tests for Phylum to Genus. P-values were adjusted where necessary using the benjamini hochberg method. All visualisation in R was performed with ggplot2 v2.2.1. Sequencing data discussed in this publication have been deposited in Sequence Read Archive (SRA) and are accessible under accession number PRJNA545405.

### qPCR

Isolated DNA was used to relatively quantify total microbial load and bifidobacteria load. Reactions were run on a 384-well LightCycler 480 PCR (Roche) using LightCycler 480 plates and adhesive cover (Roche). Each 15 μl reaction contained 6.5 μl water, 7.5 μl 2× SensiFAST™ SYBR No-ROX Master Mix (Bioline), 0.3 μl of each of 10 μM primers (forward and reverse) and 1 μl of DNA sample. No template controls (NTC) were prepared using water instead of DNA. Samples were diluted 100 times before use. Each reaction was run in quadruplicates. Primers used for total counts U16SRT-F (5-ACTCCTACGGGAGGCAGCAGT-3 (SEQ ID NO: 3), U16SRT-R (5-TATTACCGCGGCTGCTGGC-3 (SEQ ID NO: 4)), and for bifidobacteria quantification Bif-xfp-F1 (5-CGTCCGTTCTACCCGATG-3 (SEQ ID NO: 5)), Bif-xfp-R1 (5-GGTCTTCTTGCCGTCGAT-3 (SEQ ID NO: 6)). Cycling parameters were 95° C. for 5 min, followed by 45 cycles of 95° C. for 10 s, 60° C. for 30 s, and 72° C. for 30 s. A melting curve analysis (60 to 97° C.) was included at the end of every program to eliminate non-specific amplification. Crossing point (Cp) values and melting temperature were calculated automatically using instrument software. The efficiency of primers was checked using 10-fold dilutions of DNA isolated from 10 ml overnight grown culture of Bifidobacterium longum subsp. infantis ATCC 15697, resulting in E equal to 95.7% and 95.5% for U16SRT and Bif-xfp primers, respectively. The same dilution range was used as a standard curve for both microbial groups to correlate CFU/ml with Cp values. At the same time, a 16S rRNA copies/ml was calculated based on 4 copies of 16S gene in B. longum subsp. infantis ATCC 15697 genome [based on rrnDB database a mean copy number for a bacterial genome is 4.9 (https://rrndb.umms.med.umich.edu/; entry 12 Dec. 2018)].

### Metabolite Analysis

Samples were defrosted on ice and centrifuged for 1 min at maximal speed. The supernatant was filtered through a 0.2 μm filter, transferred into HPLC vessels and stored at −20° C. until measurement. Sample analysis was carried out by MS-Omics as follows: for SCFA analysis samples were acidified using hydrochloride acid, and deuterium labelled internal standards where added. All samples were analyzed in a randomized order. The analysis was performed using a high polarity column (Zebron™ ZB-FFAP, GC Cap. Column 30 m×0.25 mm×0.25 μm) installed in a GC (7890B, Agilent) coupled with a quadropole detector (5977B, Agilent). For outstanding metabolite analysis, samples were derivatized with methyl chloroformate using a protocol according to that described by Smart et al. (DOI: 10.1038/nprot.2010.108). All samples were analysed in a randomized order. The analysis was performed using gas chromatography (7890B, Agilent) coupled with a quadropole mass spectrometry detector (5977B, Agilent). In both cases, the system was controlled by ChemStation (Agilent). Raw data were converted to netCDF format using Chemstation (Agilent) before the data was imported and processed in Matlab R2014b (Mathworks, Inc.) using the PARADISe software described by Johnsen et al. in Journal of chromatography A. (2017) vol. 1503, pages 57-64.

### Results

### Microbiological Changes—16S rRNA Gene Metagenomics

DNA was isolated from samples collected during the fermentation and 16S rRNA gene amplicon sequencing was conducted. At a genus level, high diversity at the start of the fermentation in all experimental vessels was observed, representing the high diversity of the human gut microbiome. In time, the composition gradually changed in all the vessels, with the slowest changes taking place in water vessel, and fastest in Lactic acid and LACTEOL® vessels. In fact, after initial increase in Escherichia/Shigella, a dramatic increase in the relative abundance of Bifidobacterium occurred in the LACTEOL® vessel and to a lesser extent in other vessels. While the observed changes were reproducible in replicate vessels significant changes at the family level could not be detected (Wilcoxon test), most likely due to the limited number of samples per group. At the genus level, some statistically significant changes over 24 h in the water vessel were observed (Wilcoxon test), for this condition four replicates were performed and this most likely increased the power for this group. Similarly, also α-diversity visually decreased in time, as measured by Chao 1 and Shannon indexes. This drop in the α-diversity most likely reflects the natural loss of species that occurs in closed systems. However, in the event of significant overgrowth of selected taxa, a decreased α-diversity could also be observed as levels of remaining taxa may fall below the detection levels. Nonetheless, statistically significant changes in α-diversity between conditions tested at 0, 6 and 24 h were not observed as well as changes for the individual condition in time. With the exception of water vessel that had four replicates, limited number of replicates per condition may again be the cause.

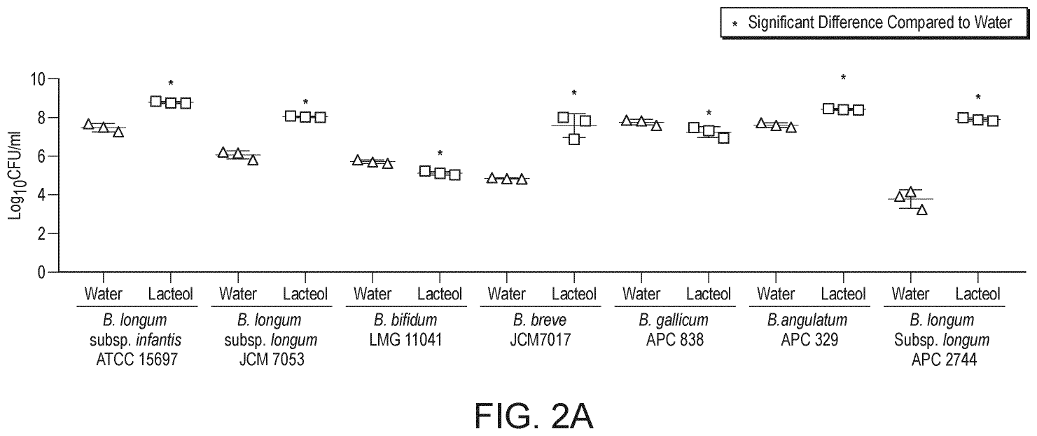

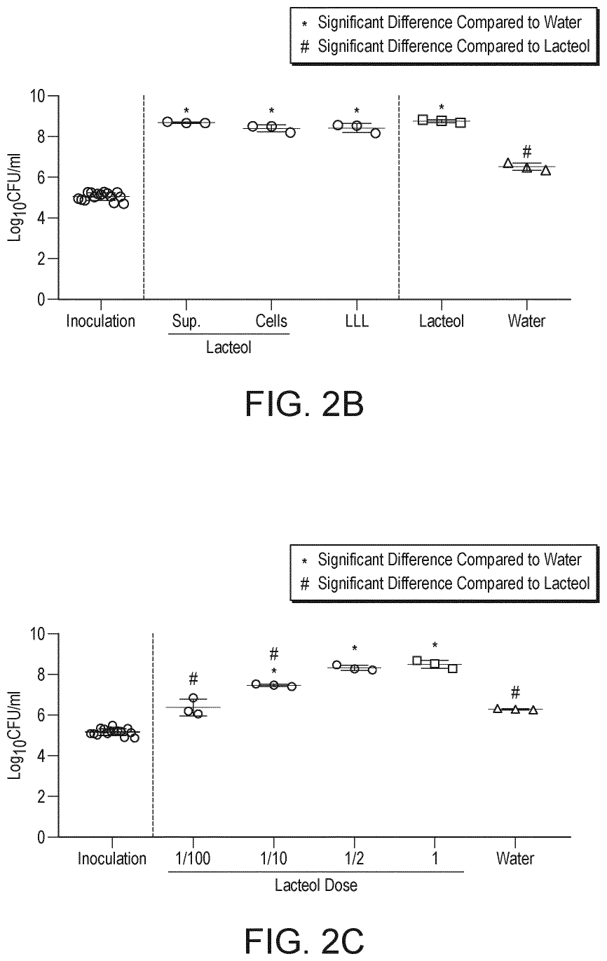

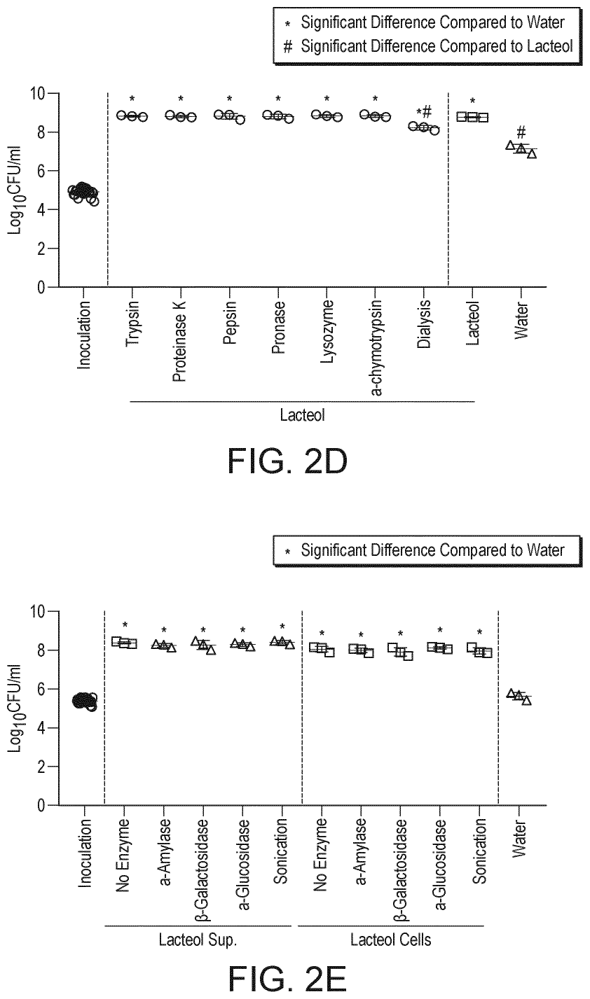

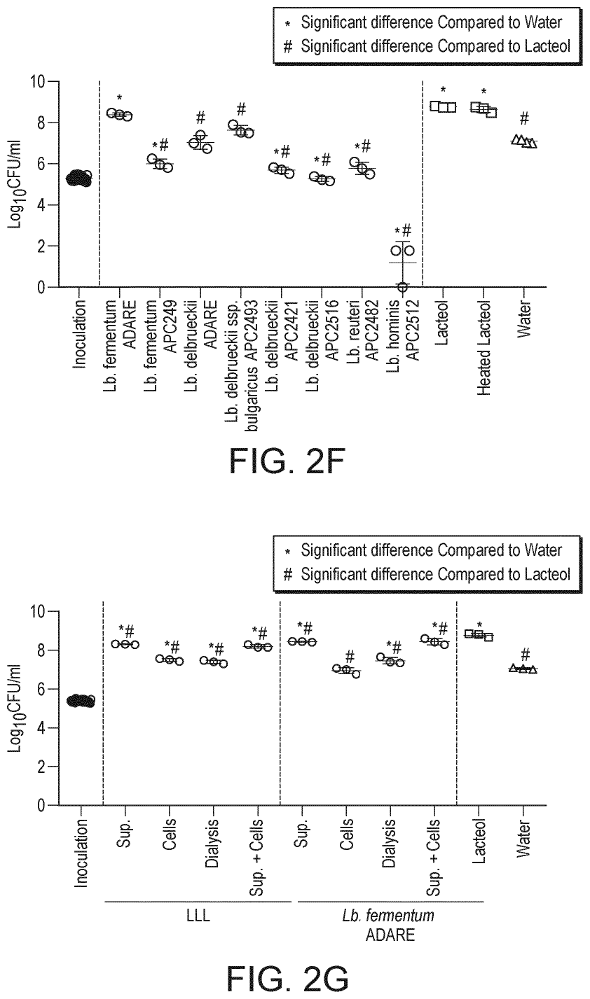

To further investigate microbiota diversity between individuals ((3 diversity) a principal coordinate analysis (PCoA) approach was used. It revealed that a clear shift in microbiota composition occurred during the 24 hours of the experiment, indicating that length of the fermentation had a major impact on the diversity with PC1 explaining 66.07% of the variation, as expected in a closed system. At the beginning all vessels clustered firmly together, again indicating that starting conditions were well standardized. At the end of the fermentation microbiota spread along the PC2 (explaining 12.44% of variation). In particular, microbiota from LACTEOL® vessel clustered at the top of the PC2 axis together with one from Lactose vessel, while the microbiota from Lactic acid vessel clustered at the lower part of the PC2 axis together with one from the water vessel (FIG. 2). No statistically significant changes in β-diversity between conditions tested at 0, 6 and 24 h as well as changes for the individual condition in time were observed. Limited number of replicates per condition may again be the cause.

Finally, analysis at the RSV level allowed more specific taxonomic differences to be assessed. Abundance of 26 RSVs was increased while 33 RSVs were reduced in LACTEOL® vessels compared to the water vessel. At the same time abundance of only three and 14 RSVs changed in Lactic acid and Lactose vessels, respectively, compared to the water vessel. In particular, six RSVs increased in the LACTEOL® vessel were assigned to genus Bifidobacterium, and only one of them, Bifidobacterium longum, could be assigned to the species level. In Lactose vessel levels of five RSVs assigned to Bifidobacterium increased compared to water vessel, with only three of them overlapping with LACTEOL® vessel.

Analysis of abundance change in time resulted in 302 RSV differently abundant in at least one condition. In particular, 36 RSVs were increased in time in all conditions while 108 RSVs were reduced. 8 RSVs were specifically changed in LACTEOL® vessels, 20 RSVs in Lactose, four RSVs in Lactic acid and 19 RSVs in water vessels.

### LACTEOL® Increases the Number of Bifidobacteria During Fermentation

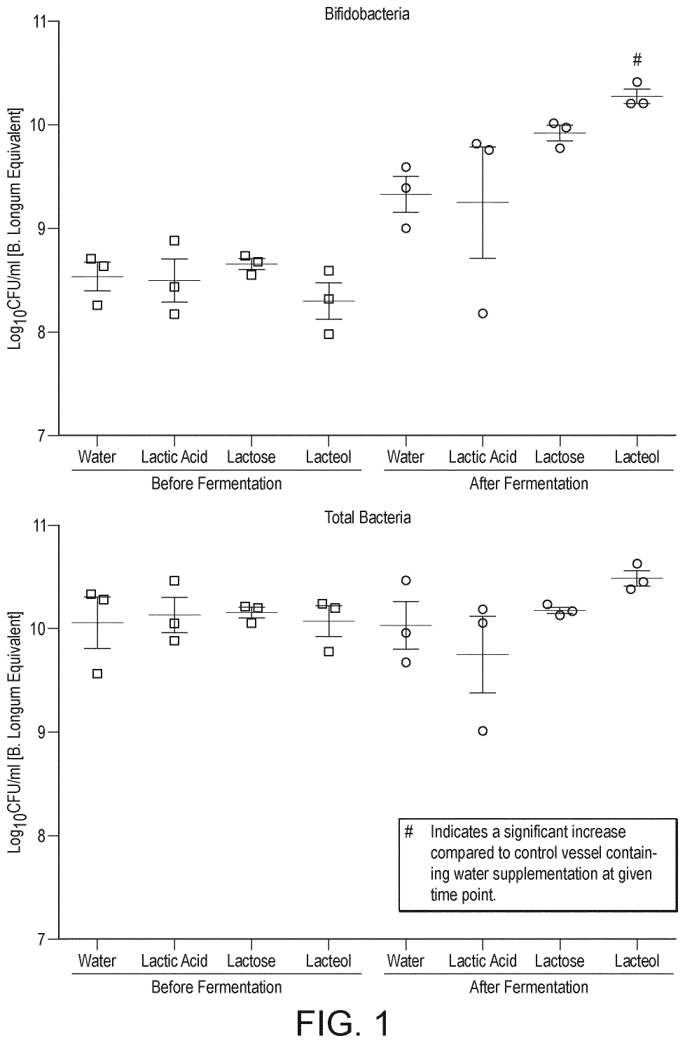

At the start of the fermentation, there was no differences in Bifidobacterium levels (One-way ANOVA; F_{3,8}=0.912, p=0.477) and total bacterial load (One-way ANOVA; F_{3,8}=0.074, p=0.972) between any of the vessels (FIG. 1). After 24 h fermentation, there were differences in Bifidobacterium levels between vessels (Kruskal-Wallis test, p=0.031) (FIG. 1). Bifidobacterium levels in water and LACTEOL® supplemented vessels differed significantly (manual post-hoc, p-value controlled for multiple comparisons, p=0.007<α/k), while other differences between all other vessels were insignificant. Also, the increase in Bifidobacterium in each vessel in time was not significant (Related Samples Wilcoxon signed Rank Test, p=0.109). At the same time, vessel supplementation had no significant effect on total bacterial load at 24 h fermentation (Welch test, p=0.108; Brown-Forsythe, p=0.285) (FIG. 1). There was also no change over time (Related Samples Wilcoxon signed Rank Test, p≥0.109).

### Metabolite Changes

Monitoring of the fermentation process revealed approximately four times higher amounts of NaOH (as well as a prolonged requirement) required to control pH in the presence of LACTEOL® compared to lactic acid or water controls. This suggests that, in presence of LACTEOL®, the fermentation process resulted in the production of higher amounts of acid and that this process was maintained for a longer time. To elucidate the nature of compounds present in in the vessels before and after fermentation metabolomics analysis was performed. Samples collected at the start of the fermentation from vessels supplemented with water, lactic acid, and lactose clustered tightly together, while samples from LACTEOL® vessel created a separate cluster. In particular, initially, 33 annotated compounds had elevated levels in the LACTEOL® vessel as compared to the water vessel. Following 24 h fermentation metabolic composition in all vessels shifted, with changes in LACTEOL® vessels being most pronounced. At this stage, 39 annotated compounds had significantly higher levels in the LACTEOL® vessel as compared to the water vessel.

### Short Chain Fatty Acids (SCFAs)

To identify the nature of the metabolites produced we focused on SCFA, and specifically, butyric acid that is known for its beneficial effects. Before the start of the fermentation there were low levels of individual SCFAs, and we observed no difference between the vessels in levels of acetic acid (Kruskal-Wallis test p=0.037; post-hoc Bonferroni p>0.05), isobutyric acid (Kruskal-Wallis test p=0.023; post-hoc Bonferroni p>0.05), and valeric acid (Kruskal-Wallis test p=1.000). In the LACTEOL® vessels we detected slightly higher levels of butyric acid (Kruskal-Wallis test p=0.030; post-hoc Bonferroni p=0.050) and propionic acid (Kruskal-Wallis test p=0.026; post-hoc Bonferroni p=0.016) compared to Lactic acid vessels, but no difference compared to water or Lactose vessels (Kruskal-Wallis test p<0.05; post-hoc Bonferroni p>0.05). Levels of formic acid differed only between Lactic acid and Lactose vessels (Kruskal-Wallis test p=0.012; post-hoc Bonferroni p=0.013). After 24 h fermentation, we observed elevated levels of acetic acid in LACTEOL® vessels compared to all other vessels. Additionally, after 24 h fermentation formic acid levels were significantly higher in LACTEOL® vessels compared to water and Lactose vessels. After fermentation, there were no significant differences between vessels in propionic acid (F(3,9)=0.382, p=0.769), butyric acid (Kruskal-Wallis test p=0.401), isobutyric acid (Kruskal-Wallis test p=0.094), and valeric acid (Kruskal-Wallis test p=0.205) levels. Levels of isovaleric acid, hexanoic acid and heptanoic acid in all samples were below the lower detection limits (LOD). Additionally, Valeric acid concentrations before fermentation were below LOD.

### TCA Cycle

Before fermentation elevated levels of succinic acid were detected in the LACTEOL® vessels (Kruskal-Wallis test p=0.018, post-hoc Bonferroni p=0.010) and fumaric acid was detected in lactose vessels (F(3,9)=5.599, p=0.019; post-hoc Bonferroni p=0.017) compared to water. As expected, levels of lactic acid were higher in Lactic acid vessels (Kruskal-Wallis test p=0.010, post-hoc Bonferroni p=0.008) compared to water. Lactic acid levels were increased in LACTEOL® vessels but did not pass the multiple comparison criteria (Kruskal-Wallis test p=0.010, pairwise comparison post-hoc Bonferroni p=0.028, multiple comparisons adjusted post-hoc Bonferroni p=0.171). A difference in pyruvate was detected between Lactic acid and Lactose vessels (Kruskal-Wallis test p=0.013, post-hoc Bonferroni p=0.019). After the 24 h fermentation levels of fumaric acid (Kruskal-Wallis test p=0.025, post-hoc Bonferroni p=0.035), succinic acid (F(3,9)=22.239, p<0.0005; post-hoc Bonferroni p<0.0005), and lactic acid (F(3,9)=120.294, p<0.0005; post-hoc Bonferroni p<0.0005) were elevated in LACTEOL® vessels compared to water. Levels of lactic acid were also elevated in Lactic acid (F(3,9)=120.294, p<0.0005; post-hoc Bonferroni p=0.001) and Lactose (F(3,9)=120.294, p<0.0005; post-hoc Bonferroni p<0.0005) vessels compared to water but not to the levels found in LACTEOL® vessels (post-hoc Bonferroni p<0.0005 for both). There was no difference between the vessels, before and/or after the fermentation, in measurements on the remaining six TCA cycle compounds (Kruskal-Wallis test p>0.05).

### Amino Acids

Before fermentation 12 of 19 tested amino acids had elevated levels in LACTEOL® vessels compared to water vessels. In particular, tryptophan levels were higher in LACTEOL® vessels compared to all other vessels (F(3,9)=17.431, p<0.0005; post-hoc Bonferroni p<0.006). After fermentation ten of 19 tested amino acids had elevated levels in LACTEOL® vessels compared to water vessels.

### Other Metabolites

Before fermentation 20 of 61 tested identified and annotated compounds had elevated levels in LACTEOL® vessels compared to water vessels. After fermentation 26 of 61 tested identified and annotated compounds had elevated levels in LACTEOL® vessels compared to water vessels.

### Conclusion

Example 1 investigated the impact of LACTEOL® upon microbiota composition in anaerobic batch cultures inoculated with human fecal samples. LACTEOL® is known to contain lactic acid that is generated during initial fermentation by L. fermentum and L. delbrueckii as well as lactose that is added post-production to facilitate the lyophilization process. Therefore as additional controls besides water, we included supplementation with lactic acid and lactose individually. Both microbiome and metabolite analysis indicated a tight clustering of samples collected before fermentation confirming, on the one hand, the reproducibility of the preparation, and on the other hand, the high composition diversity of the human gut microbiome. As expected we saw no differences between vessels in regards to the microbiome. However, LACTEOL® vessels showed altered metabolite profiles, predominantly in terms of elevated levels of amino acids, setting this condition apart from the controls.

In time both microbiome and metabolite profiles in all vessels shifted, with changes in LACTEOL® vessels being most pronounced. LACTEOL® supplementation increased both relative as well as absolute abundance of Bifidobacterium during the 24 h fermentation. In line with this expansion in the LACTEOL® vessels, we observed increased levels of acetic acid, formic acid and lactic acid, previously shown to be produced by bifidobacteria. Presence of either bifidobacteria or SCFA has been generally considered beneficial.

### Example 2

### LACTEOL® Preparations

In growth experiments reconstituted LACTEOL® powder (0.34 g/ml) was used. Alternatively, Low Lactose LACTEOL® (LLL) was used. Each preparation containing 5×10⁹ to 1×10¹⁰ cell bodies per ml of the solution unless stated otherwise.

### Preparation of Bifidobacterium Inoculum

100 μl of −80° C. stock of a Bifidobacterium strain (Table 1) was injected into anaerobic Hungate tubes containing 10 ml of MRS broth (Difco, BD) supplemented with L-cysteine (final concentration 0.6 g/L) and rezuzarin (final concentration 1 mg/L). Tubes were incubated overnight at 37° C. 100 μl of 100 times diluted overnight culture was used as inoculum unless stated otherwise. For strains B. bifidum LMG 11041 and B. gallicum APC 838 100 μl of 10 times diluted overnight culture was used due to the poor overnight growth.

TABLE 1
Strains used in this study

| Strain | Isolation | Use |
|---|---|---|
| Lactobacillus fermentum CNCM MA64/4E-1b | Healthy human faeces | Producer |
| Lactobacillus delbrueckii CNCM MA64/4E-2z | Healthy human faeces | Producer |
| Limosilactobacillus reuteri APC2482 (DSM20016, ATCC 23272) | Intestine of adult | Comparison |
| Lactobacillus delbrueckii ssp. bulgaricus APC 2493 (DSM20081, ATCC 11842) | Bulgarian yoghurt | Comparison |
| Lactobacillus delbrueckii APC 2421 (DSM20074, ATCC 9649) | Sour grain mash | Comparison |
| Lactobacillus delbrueckii APC 2516 (DSM 20072, ATCC 12315) | Emmental cheese | Comparison |
| Limosilactobacillus fermentum APC249 (ATCC 14931, DSM 20052) | Fermented beets | Comparison |
| Lactobacillus hominis APC 2512 (DSM23910) | Human intestine | Comparison |
| Bifidobacterium longum ssp. infantis ATCC 15697 (B1) | Intestine of infant | Main Target |
| Bifidobacterium longum subsp. longum JCM 7053 (B2) | Infant faeces | Target |
| Bifidobacterium bifidum LMG 11041 (ATCC29521) (B3) | Infant faeces | Target |
| Bifidobacterium breve JCM7017 (B4) | Human faeces | Target |
| Bifidobacterium gallicum APC 838 (ATCC 49850) (B5) | Human faeces | Target |
| Bifidobacterium angulatum APC 329 (ATCC 27535) (B7) | Human faeces | Target |
| Bifidobacterium longum subsp. longum APC 2744 (ATCC 15707) (B8) | Intestine of adult | Target |
| Enterococcus faecium DPC1146 | National Dairy Products | Non-target |
| | Research Centre collection | |
| Escherichia coli K12 | Stool of a convalescent | Non-target |
| | diphtheria patient | |

### Bifidogenic Assay

9 ml of base medium (10× or 15× diluted; dilution factor set for each batch of powdered media required for no or minimal growth of Bifidobacterium when supplemented with water) MRS broth supplemented with L-cysteine (final concentration 0.6 g/L) and resazurin (final concentration 1 mg/L)) in an anaerobic Hungate tube was supplemented with 1 ml of test solution (LACTEOL® preparation) or one of the controls (predominantly water). Next, the Hungate tube was inoculated and the TO sample was collected using a syringe and needle to limit oxygen access. The Hungate tube was incubated at 37° C. and at regular intervals, samples were taken. Collected samples were serially diluted in PBS and 100 μl were plated in duplicate on fresh MRS agar plates supplemented with L-cysteine (final concentration 0.6 g/L). Plates were incubated for at least 48 h at 37° C. in jars with anaerobic generators (BioMerieux) before colony counting.

### LACTEOL® Treatments

### (a) Enzymatic Treatments

5 ml of LACTEOL® solution was supplemented with 10 mg of Proteinase K (with the addition of 1 mM CaCl₂·2H₂O), Trypsin, Pepsin, Pronase, Lysozyme, or α-chymotrypsin. 5 ml cell and supernatant fractions of LACTEOL® solution were supplemented with 1000 units cellulase, 25 units α-glucosidase, 1000 units α-amylase, and 125 units β-galactosidase. Tubes were incubated for 4 h at 37° C. with shaking followed by 1 h at 92° C. Enzymatically treated LACTEOL®, cells, and supernatant fractions were stored at 4° C. until tested in the bifidogenic assay. The pH of supernatants treated with α-amylase and β-galactosidase was increased to 7.16 before enzyme addition and adjusted back to the original pH and filtered after incubation. 1 ml of enzymatically treated cells or supernatant were used in the bifidogenic assay (10× diluted media).

### (b) Physical Treatments

### Dialysis

5.1 g of LACTEOL® resuspended in 15 ml water was transferred into a washed 1 kDa dialysis tube (Pur-A-Lyser Magna 1000, Sigma). The tube was then placed in 4.5 L of demineralized water and incubated with steering at 4° C. for 4 days. Water was changed daily. The content of the Tube was transferred into a Hungate tube and stored at 4° C. until use in the bifidogenic assay (10× diluted media).

### Sonication

LACTEOL® solution was centrifuged, the supernatant was filtered, and the cell pellet was washed twice. Supernatant and cell fractions were sonicated for 4 h and stored at 4° C. until use in the bifidogenic assay (10× diluted media).

### Alternative Treatments—Probiotics

### Preparation of Lab-Made Lactobacilli Preparations

Lactobacillus strains (Table 1) were streaked from −80° C. stocks onto MRS plates. 10 ml MRS broth was inoculated with a single colony and incubated anaerobically overnight at 37° C. 1% inoculum was used for flasks with MRS broth supplemented with L-cysteine (final concentration 0.6 g/L). Media for growth of Lb. delbrueckii 2z was supplemented with 1% Pepsin from casein to facilitate its growth requirements. Following anaerobic overnight incubation at 37° C. content of flasks was distributed into large Petri dishes and placed in −80° C. until freeze-drying. Freeze-dried content was scraped off the plates and resuspended to 0.34 g/ml water before heat treatment for 1 h at 110° C. (heat treatment applied during LACTEOL® preparation) and stored at 4° C. until use.

### Effect of Market Products on Bifidobacteria

Solutions of market products were prepared in concentrations corresponding to their daily dose. In particular, the content of one capsule of Culturelle (10 billion cells of Lacticaseibacillus rhamnosus GG and inulin) was resuspended in 1 ml of water. Contents of three flacons of Enterogermina (SANOFI; 6 billion spores of Bacillus clausii SIN, B. clausii 0/C, B. clausii T, and B. clausii N/R) were centrifuged (10 min at 4696×g) and resuspended in 1 ml of water. Five drops of BioGaia (10⁸ CFU of Limosilactobacillus reuteri DSM 17938) were resuspended in 1 ml of water. The content of one capsule of Ultra Levure (BIOCODEX; 200 mg of Saccharomyces boulardii CNCM 1-745) was resuspended in 1 ml of water. The equivalent of one capsule of LACTEOL® (340 mg, 10 billion cells of Lb. fermentum and Lb. delbrueckii) was resuspended in 1 ml of water. 1 ml of market product solution was used to test its effect in the modified bifidogenic assay (15× diluted media). Serially diluted samples were plated on MRS plates with L-cysteine (0.6 g/L), cycloheximide (70 mg/L), and mupirocin (50 mg/L). Selective enumeration of product counts was performed for Lb. rhamnosus GG (subtracting bifidobacteria counts from MRS counts), B. clausii (plating on BHI, aerobic incubation), Lb. reuteri DSM 17938 (MRS+Tetracycline (3011 g/mL), anaerobic), and Saccharomyces boulardii CNCM 1-745 (Saboraud (4% dextrose), aerobic incubation).

### LACTEOL® Purification

### Solid Phase Extraction C18 Column

In all C18 purifications a half strength of LACTEOL® preparation (equivalent to 1.7 g in 10 ml) was used as this was the maximum binding capacity of the 10 g Strata C18 Solid Phase Extraction column (Phenomenex). Consequently, flow, wash, and elution fractions were half strength unless stated otherwise. Half strength LACTEOL® was centrifuged for 5 min at 5000 rpm (Servall ST 16R, with rotor TX-400). The resulting supernatant was filtered through a 0.2 μm filter to remove solid particles. 10 g Strata C18 Solid Phase Extraction column was connected to a vacuum pump and conditioned with 120 ml of methanol and equilibrated with 120 ml of HPLC water. The filtered supernatant was applied on the column and gravitationally allowed to pass through, resulting flow-through was collected. Next, the column was washed with 120 ml of 5% methanol, the wash was collected. Following 5 mins of vacuum drying of the column, 120 ml of methanol was applied to the column to allow elution of the fractions still bound to the sorbent, elution was collected. Collected wash and elution fractions were rotary evaporated to remove solvents and concentrate samples to the initial concentration in water. Flow-through, wash, and elution fractions were filter-sterilized (0.2 μm filter) and stored at 4° C. until use.

### Ammonium Sulfate Precipitation

Low lactose LACTEOL® (LLL) solution (equivalent to 3.4 g in 10 ml) was centrifuged for 10 min at 5000 rpm (equipment details). The resulting supernatant was filtered through a 0.2 μm filter to remove solid particles. Gradually 3.18 g of ammonium sulphate (aiming to reach 50% saturation) was dissolved at room temperature in 10 ml of the supernatant in duplicate. One flask was incubated for 6 h, while the second flask was incubated for 1 h, at room temperature with shaking. Next, flask contents were centrifuged for 15 min, 5000 rpm at 4° C. The supernatant from the second flask was supplemented with ammonium sulphate to reach 60% saturation and incubated as previously. This approach was continued until reaching 80% saturation (ammonium sulphate required to reach 90% saturation did not dissolve). Precipitates collected at different stages were dissolved in 10 ml of buffer (150 mM Tris, 150 mM NaCl, pH 7.5) and stored at 4° C. until desalting using a C18 column (see above).

### HPLC Analysis

### HPLC Analysis of Raw Samples

Supernatant samples were diluted 1 in 2 (LACTEOL® and LLL) and 1 in 7 (Lb. fermentum and Lb. delbrueckii) in Milli Q water. 100 μl aliquots were applied to a Toso Haas gel permeation column (TSK gel G2000SW and G2000SWXL in series, 511, 7.8×30 cm) running a 30% acetonitrile isocratic gradient at 1 ml/min. The HPLC eluent is monitored by UV absorption at 214 nm and fractions collected at 1-minute intervals over 35 min for assay.

### HPLC Analysis of C18 Purified Sample

27×80 μl aliquots (2160 μl in total) of the supernatant sample were applied to a Toso Haas gel permeation column (TSK gel G2000SW and G2000SW_{XL}, in series, 511, 7.8×30 cm) running a 30% acetonitrile 0.1% TFA isocratic gradient at 1 ml/min over 35 minutes. The HPLC eluent is monitored by UV absorption at 214 nm and fractions collected at 1-minute intervals. Fractions 37-60 were tested in MALDI TOF Mass Spectrometry and following centrifugal concentration were tested in the bifidogenic assay.

### Testing of Non-Bifidobacteria Strains

−80° C. stock of E. faecium and E. coli were streaked on tryptone soy agar (TSA, producer) and Luria-Bertani (LB, producer) plates, respectively and incubated overnight at 37° C. A single colony was used to inoculate 20 ml of either TSB or LB broth, followed by overnight incubation at 37° C. Anaerobic Hungate tube containing either 9 ml of 1.1×MRS broth (0.6 g/L L-cysteine, 1 mg/L resazurin) or dilution range of LB (in water or PBS) or PBS were supplemented with 1 ml water or 1 ml of LACTEOL® solution and inoculated with 100 μl of 100 times diluted overnight cultures. Anaerobic tubes were incubated overnight at 37° C. The collected samples we serially diluted and plaited either on TSA or LB plates followed by overnight aerobic incubation at 37° C.

### Results

### Effect of LACTEOL® and LACTEOL®-Like Preparations on Growth of Selected Bifidobacteria

LACTEOL® stimulated growth of both infant-associated and adult-associated bifidobacteria in pure culture. Specifically, Bifidobacterium longum subsp. infantis ATCC 15697 (B1; t₄=18.555, p<0.0005), Bifidobacterium longum subsp. longum JCM 7053 (B2; t₄=15.626, p=0.003), Bifidobacterium bifidum LMG 11041 (ATCC29521) (B3; t₄=−7.432, p=0.002), Bifidobacterium breve JCM7017 (B4; t₄=7.667, p=0.016), Bifidobacterium gallicum APC 838 (ATCC 49850) (B5; t₄=−2.828, p=0.047), Bifidobacterium angulatum APC 329 (ATCC 27535) (B7; t₄=12.065, p<0.0005), and Bifidobacterium longum subsp. longum APC 2744 (ATCC 15707) (B8; t₄=14.603, p<0.0005) (FIG. 2A). In more detail, LACTEOL® stimulated the growth of B. longum subsp. infantis ATCC 15697 in pure culture (ANOVA; F_{4,10}=108.650, p<0.0005; Bonferroni post-hoc, p<0.0005) (FIG. 2B). At the same time, both soluble (supernatant) and insoluble (mainly cells) fractions of LACTEOL® stimulated the growth of B. longum subsp. infantis ATCC 15697 to the same extent as LACTEOL® (Bonferroni post-hoc, compared to water all p<0.0005, compared to LACTEOL® p=1, p=0.165, respectively) (FIG. 2B). The low lactose version of LACTEOL® also showed comparable growth stimulation to full LACTEOL® ((Bonferroni post-hoc, compared to water p<0.0005, compared to LACTEOL® p=0.225) (FIG. 2B).

Growth of Bifidobacterium depended on the LACTEOL® dose (FIG. 2C; ANOVA; F_{5,11}=59.654, p<0.0005). Half strength LACTEOL® stimulated growth to the same extent as LACTEOL® (Bonferroni post-hoc, p=1), while Bifidobacterium counts in 100 times diluted LACTEOL® were the same as in water control (Bonferroni post-hoc, p=1). The LACTEOL® activity was not affected by enzymatic treatment and heat treatment normally used to inactivate enzymes (1 h at 92° C.). LACTEOL® preparations treated with a range of proteolytic enzymes (FIG. 2D) as well as mock-treated LACTEOL® (FIG. 2D) stimulated the growth of B. longum subsp. infantis ATCC 15697 in pure culture (ANOVA; F_{8,18}=73.582, p<0.0005; Bonferroni post-hoc compared to water, all p<0.0005; Bonferroni post-hoc compared to LACTEOL®, all p=1) (FIG. 2D). Supernatants and cell fractions of LACTEOL® preparation treated with carbohydrate digesting enzymes (FIG. 2E) as well as their mock-treated versions (FIG. 2E) stimulated the growth of B. longum subsp. infantis ATCC 15697 in pure culture (ANOVA; F_{10,22}=85.783, p<0.0005; Bonferroni post-hoc compared to water, all p<0.0005; Bonferroni post-hoc compared to supernatant or cell fraction of LACTEOL®, all p>0.05) (FIG. 1E). Interestingly, 1 kDa dialyzed LACTEOL® also stimulated the growth of B. longum subsp. infantis ATCC 15697 in pure culture (FIG. 2D; ANOVA; F_{8,18}=73.582, p<0.0005; Bonferroni post-hoc compared to water, p<0.0005) but not to the level of LACTEOL® (ANOVA; F_{8,18}=73.582, p<0.0005; Bonferroni post-hoc compared to LACTEOL®, p<0.0005). Sonication did not affect the activity of ether supernatant or cell fraction of LACTEOL® (FIG. 2E; ANOVA; F_{10,22}=85.783, p<0.0005; Bonferroni post-hoc compared to water, all p<0.0005; Bonferroni post-hoc compared to supernatant or cell fraction of LACTEOL®, all p=0.05). There was a difference in 24 h growth levels in response to LACTEOL®-like preparations (FIG. 2F; ANOVA; F_{10,21}=141.368, p<0.0005). LACTEOL® and Lb. fermentum 1b comparably stimulated Bifidobacterium growth (Bonferroni post-hoc, compared to each other p=1, compared to water p<0.0005), while Lb. fermentum APC249, Lb. delbrueckii APC2421, Lb. delbrueckii APC2516, Lb. reuteri APC2482 did not promote growth (Bonferroni post-hoc, all p<0.0005). Lb. hominis APC2512 supplementation had a clear killing effect on Bifidobacterium as six or fewer CFUs were recovered on plates. Lb. delbrueckii 2z and Lb. delbrueckii ssp. bulgaricus APC2493 had the same effect as water supplementation (Bonferroni post-hoc, p=1 and p=0.060, respectively).

For both LLL and Lb. fermentum 1b, supernatants and full preparations (containing supernatant and cells) equally stimulated growth of Bifidobacterium (FIG. 2G; ANOVA; F_{9,20}=113.988, p<0.0005; Bonferroni post-hoc, p>0.514), but not to the same level as LACTEOL® (Bonferroni post-hoc, p<0.039). LLL cells (but not Lb. fermentum 1b cells) and non-dialyzed components of LLL and Lb. fermentum 1b showed comparable intermediary Bifidobacterium growth stimulation (Bonferroni post-hoc, compared to each other p=1, compared to water and LACTEOL® p<0.036).

### Effect of Commercial Probiotic Preparations on Bifidobacteria Growth

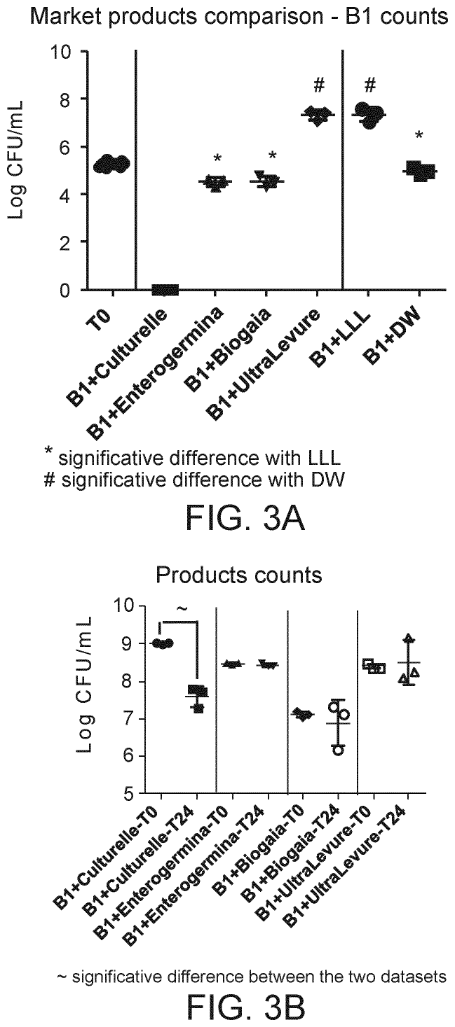

Before incubation with commercial products there was no difference between the conditions (ANOVA; F_{5,12}=0.627, p=0.683). After the 24 h incubation, bifidobacteria counts differed significantly (ANOVA; F_{5,12}=574.535, p<0.0005). Supplementation with Culturelle prevented the recovery of bifidobacteria (Bonferroni post-hoc compared to any other treatment, p<0.0005). Both Enterogermina and BioGaia had no impact on bifidobacteria counts, with counts comparable with water (Bonferroni post-hoc compared to water, p=0.192 and p=0.242, respectively). Finally, Ultra Levure stimulated bifidobacteria growth to a comparable level as LLL (Bonferroni post-hoc compared to water, p<0.0005; Bonferroni post-hoc compared to Low Lactose LACTEOL®, p=1) (FIG. 3A). Over the 24 h there was no change in numbers of the microorganisms composing the commercial products: Culturelle (Wilcoxon test, p=0.109), Enterogermina (t₂=1.941, p=0.192), BioGaia (t₂=0.595, p=0.612), and Ultra Levure (t₂=−0.253, p=0.824) (FIG. 3B).

### Purification of LACTEOL® Preparations

### SPE C18 Purification

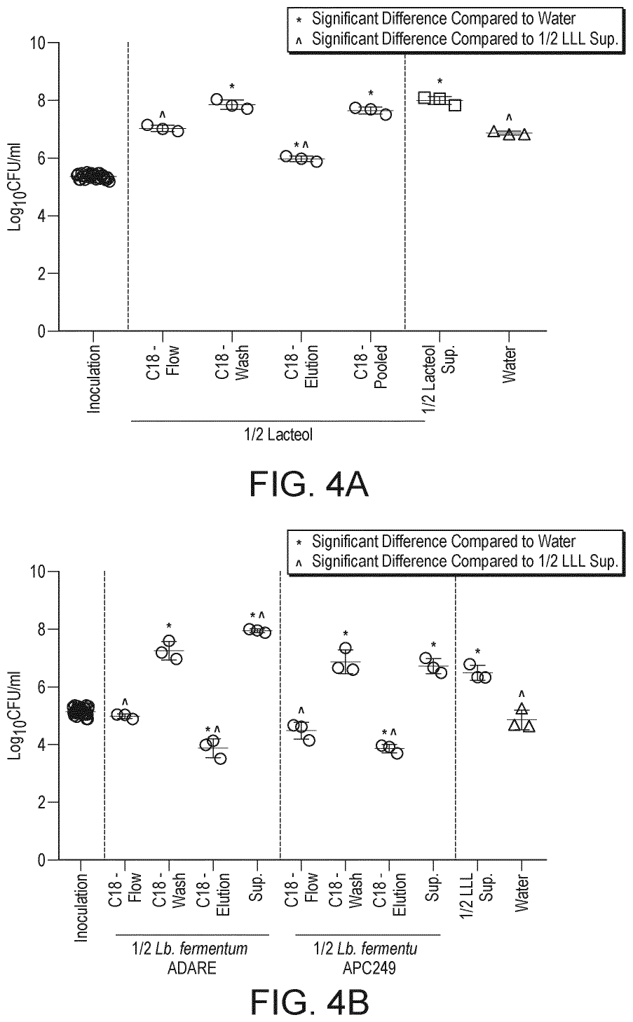

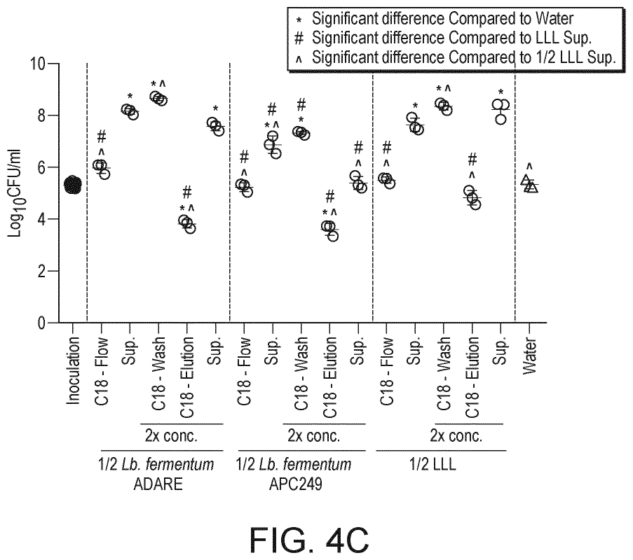

Processing half strength LACTEOL® supernatant with an optimized purification protocol for C18 columns resulted in differences in bifidobacteria CFU counts for the resulting fractions (FIG. 4A; ANOVA, F_{5,12}=117.546, p<0.0005). All fractions except for the flow-through (Bonferroni post-hoc p=1), showed significant differences compared to the water control (Bonferroni post-hoc p<0.0005). Wash and pooled fractions showed no difference compared to half strength LACTEOL® supernatant (Bonferroni post-hoc p=1 and p=0.066, respectively), indicating that the active fraction is present in the wash fraction. Wash fractions from C18 purification of both half strength Lb. fermentum 1b (FIG. 4B; ANOVA, F_{5,12}=110.087, p<0.0005; Bonferroni post-hoc p=0.053) and half strength Lb. fermentum APC249 (FIG. 4B; ANOVA, F_{5,12}=57.473, p<0.0005; Bonferroni post-hoc p=1) showed the same level of Bifidobacterium growth stimulation as half strength LLL supernatant (as well as their own half strength supernatants), while flow and elution fractions showed no growth stimulation. When testing full strength wash fraction of Lb. fermentum 1b (twice concentrated wash fraction of half strength Lb. fermentum 1b) we observed the same Bifidobacterium growth stimulation as with LLL (FIG. 4C; ANOVA, F_{15,32}=189.939, p<0.0005; Bonferroni post-hoc p=1) and half strength Lb. fermentum 1b (Bonferroni post-hoc p=0.700) supernatants. While half strength Lb. fermentum APC249 supernatant did stimulate Bifidobacterium growth (albeit not to the level of full or half strength LLL and Lb. fermentum APC249), the full Lb. fermentum APC249 supernatant did not stimulate Bifidobacterium growth (Bonferroni post-hoc compared to water p<0.0005 and p=1, respectively), indicating the presence of concentration-dependent bifidostatic compound(s) in Lb. fermentum APC249 supernatant. However, with the full strength wash fraction of Lb. fermentum APC249 (twice concentrated wash fraction of half strength Lb. fermentum APC249) the same Bifidobacterium growth stimulation was seen as with half strength LLL supernatant (Bonferroni post-hoc p=1) suggesting that the bifidostatic compound(s) are at least partially removed during the C18 purification process.

### Ammonium Sulphate Precipitation

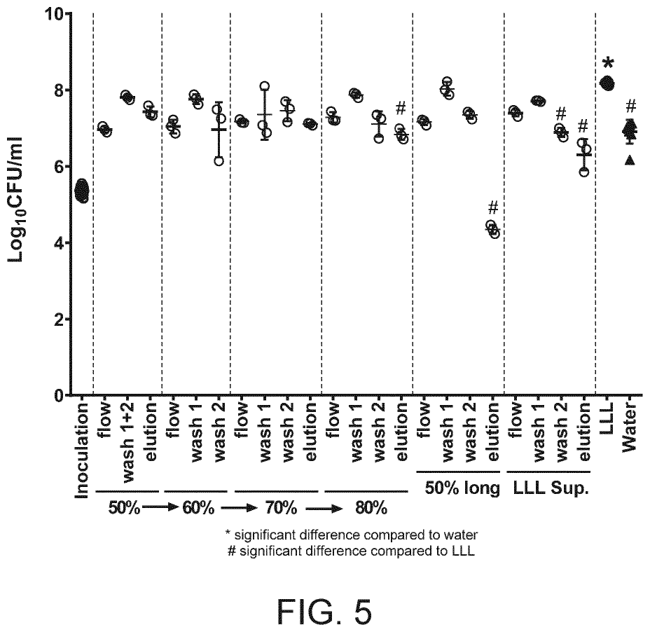

Differences in counts after 24 h growth of Bifidobacterium were observed (FIG. 5; Independent samples Kruskal-Wallis, χ² (23)=69.998, p<0.0005). Wash 1 fractions from C18 clean-up of ammonium sulphate precipitates consistently gave the highest Bifidobacterium counts. Each stepwise increase in ammonium sulphate concentration resulted in preparations showing stimulation of Bifidobacterium growth comparable to LLL and water.

### HPLC Analysis of LACTEOL® and its Fractions

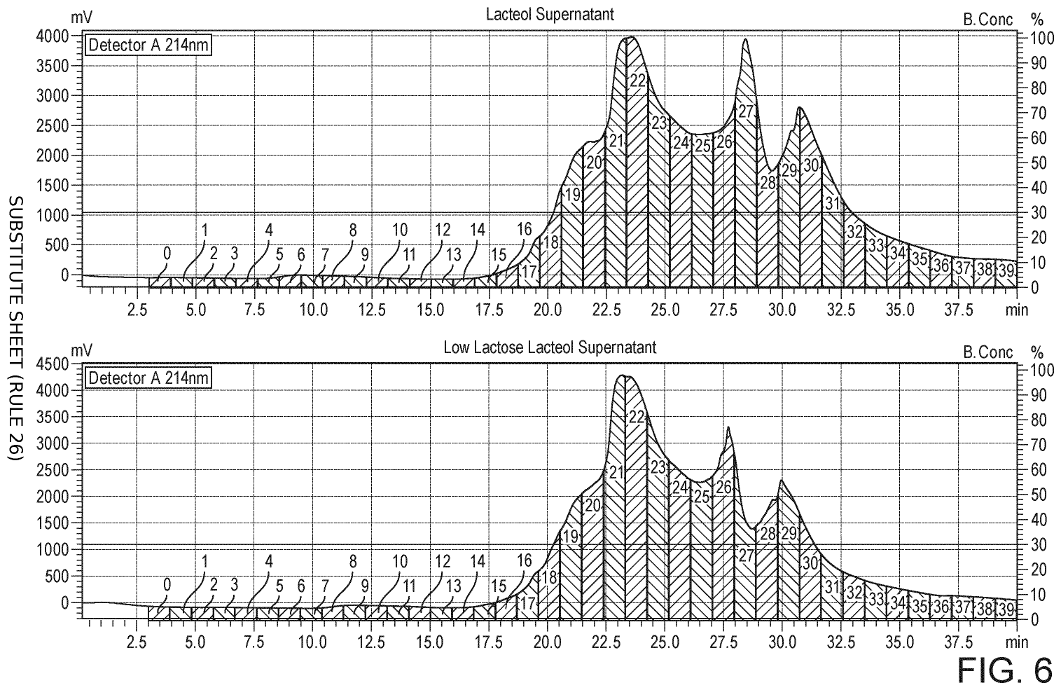

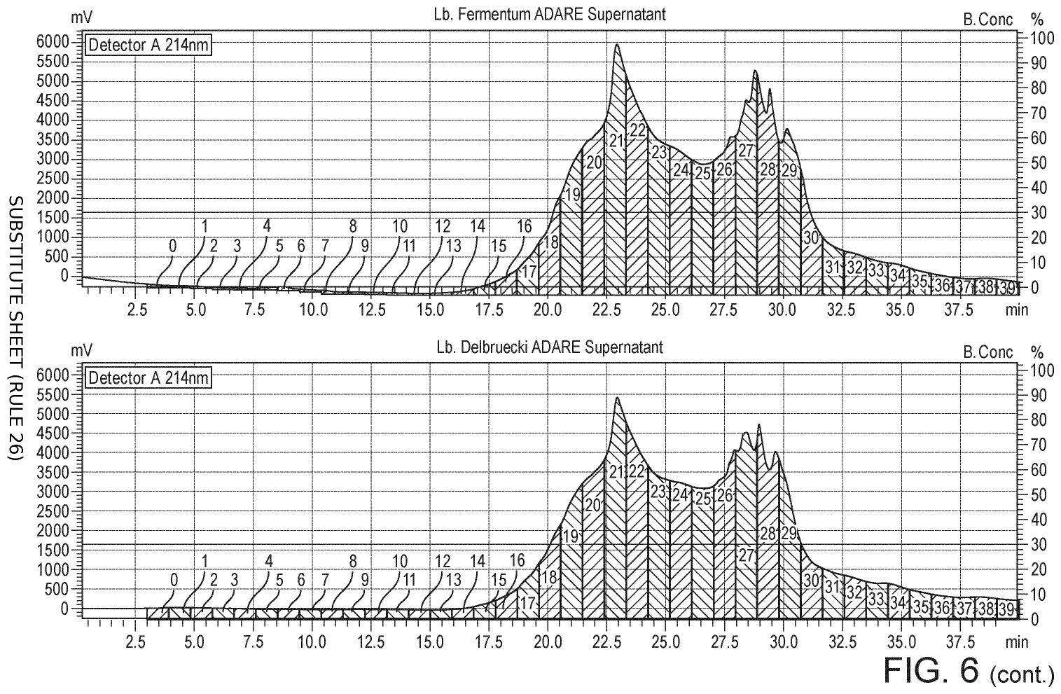

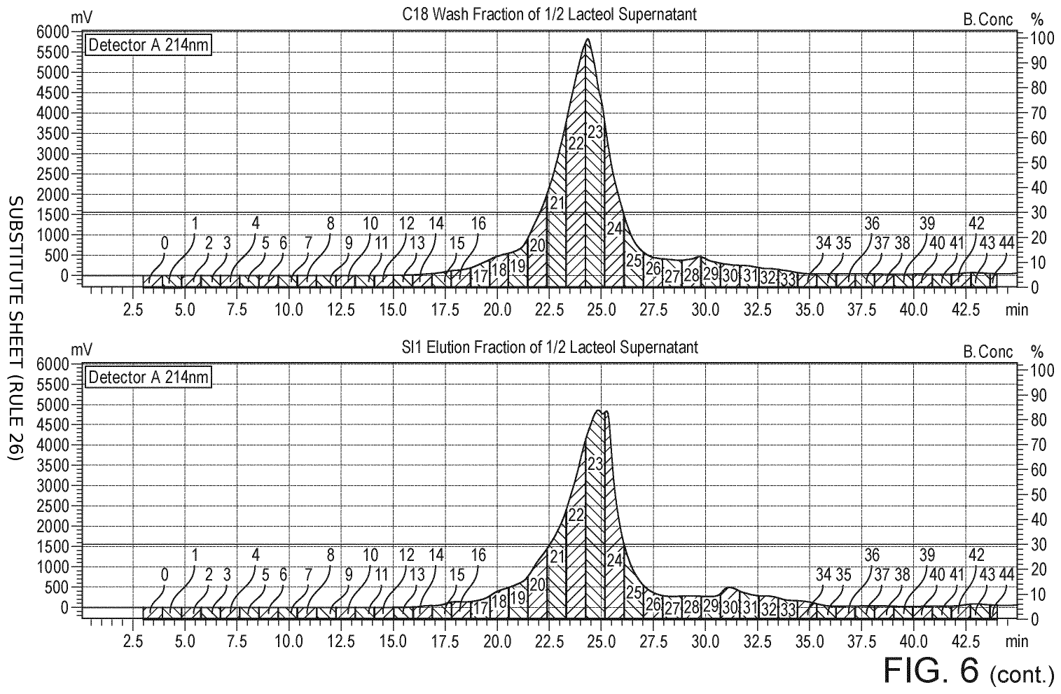

UV absorption profiles of LACTEOL®, LLL and supernatants of two producer strains confirmed the highly diverse composition of the preparations. The SPE purification using either C18 or S11 columns removed a large proportion of compounds with no impact on bifidobacteria growth, resulting in simpler UV absorption peaking around 23 min (FIG. 6).

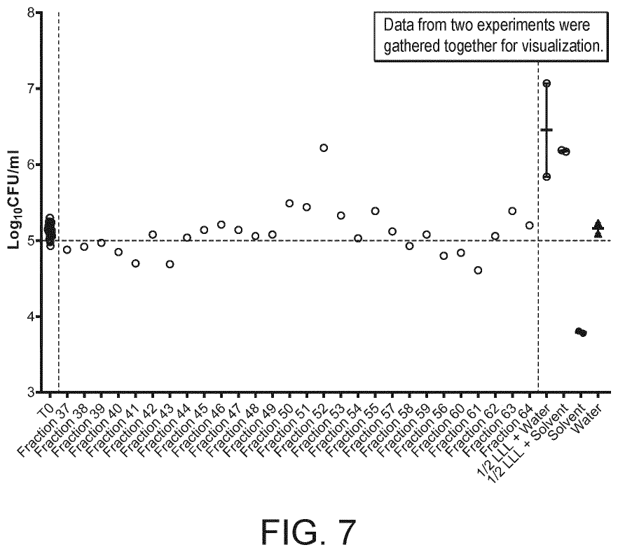

Fractions collected during multiple HPLC runs with C18 purified half-strength LACTEOL® following concentration were tested in the bifidogenic assay. Fraction 52 showed promising results as it had higher Bifidobacterium counts. However, this was a single replicate with negative controls (concentrated HPLC solvent) showing decreased Bifidobacterium levels (FIG. 7).

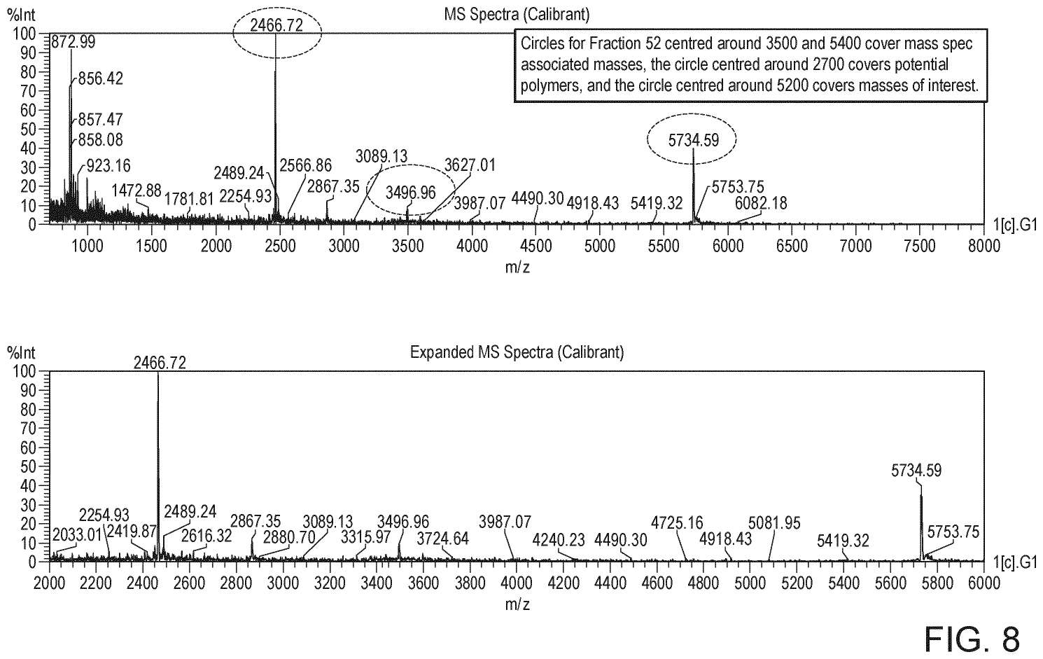

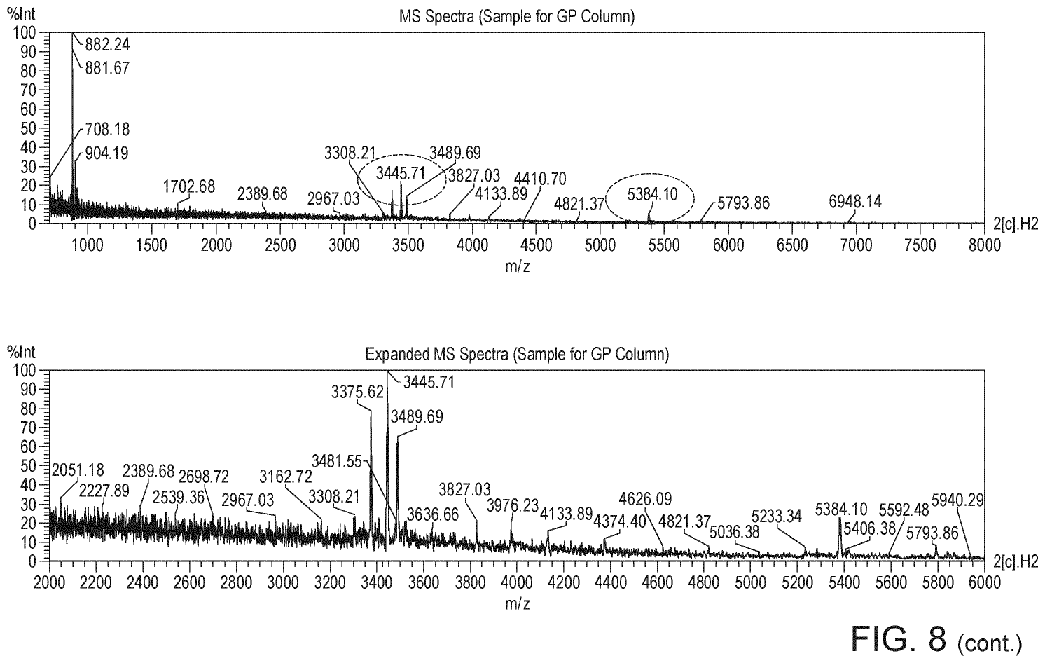

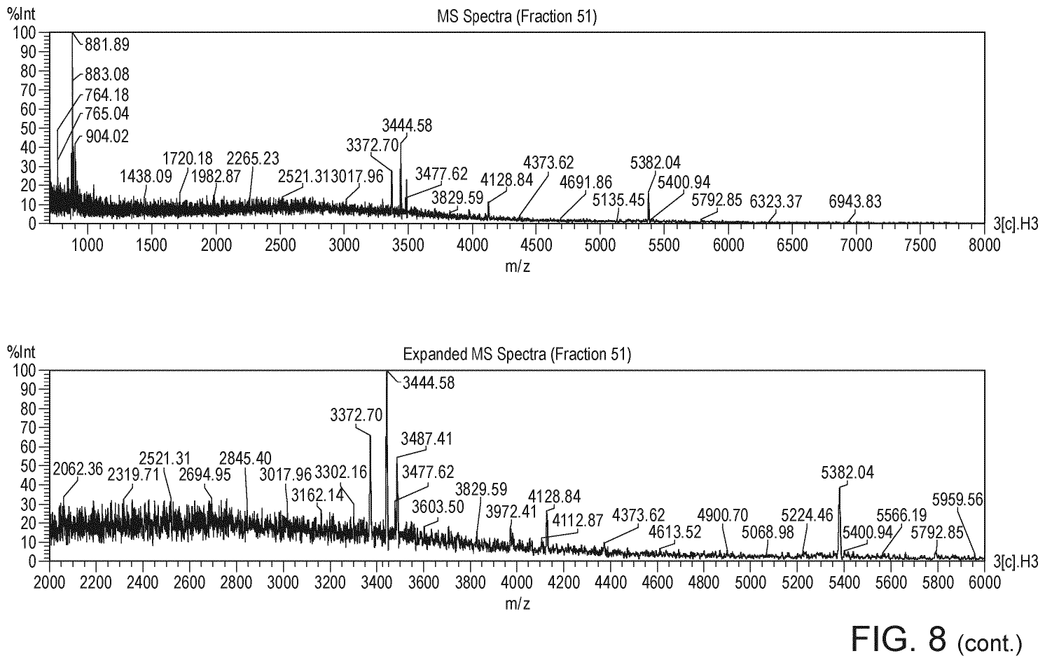

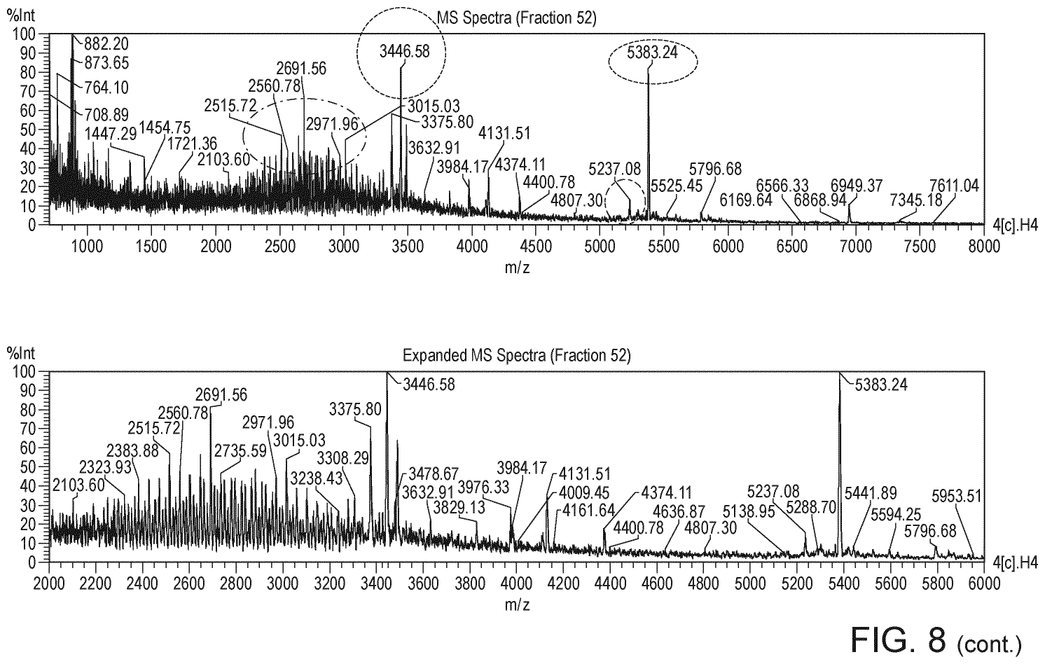

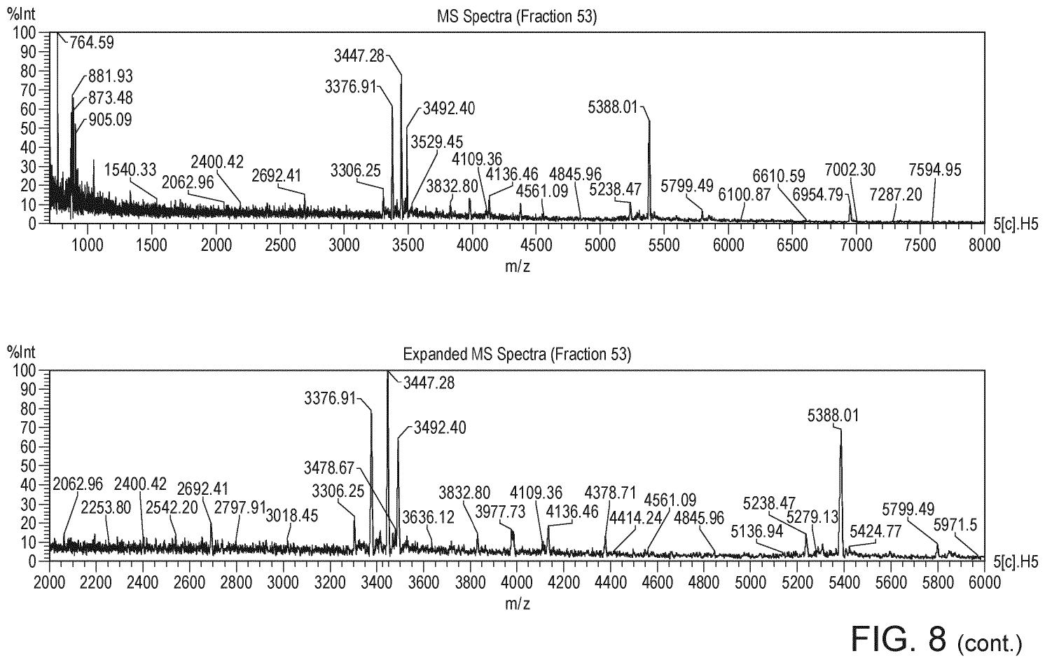

MALDI TOF mass spectrometry revealed similar profiles in fractions 51, 52, and 53, with most pronounced peaks coming from mass spec measurement set up rather than from the measured samples. In faction 52, we saw an increased occurrence of masses ranging from 2500 to 3000 m/z, reflecting the presence of undefined molecules. A single peak at a 5237.08 m/z was present in fraction 52, but not in neighboring fractions (FIG. 8).

### Concentration-Dependent Effect of LACTEOL®-Like Preparations on Bifidobacteria Growth

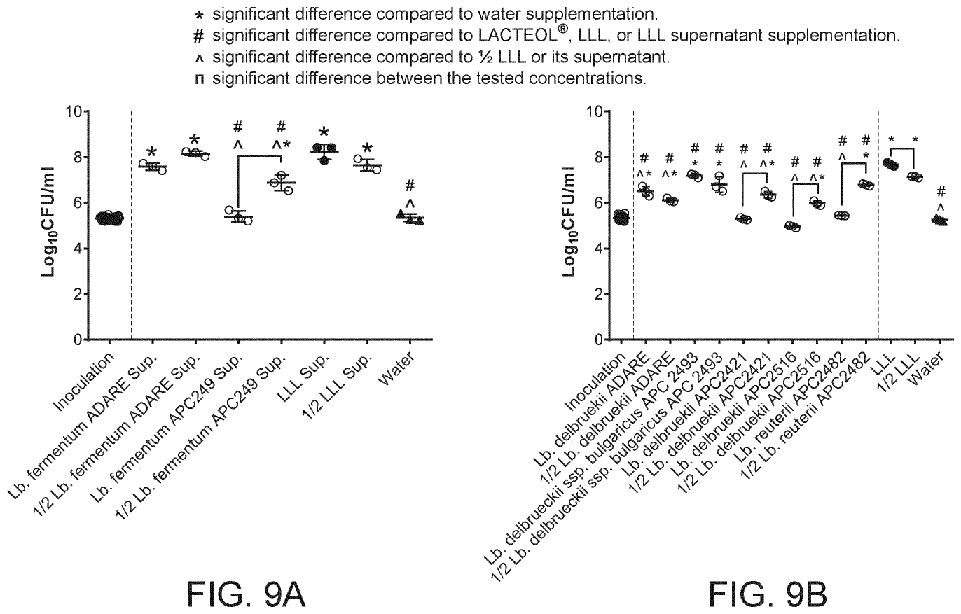

Differences were observed when testing supernatants of the LACTEOL®-like preparation (FIG. 9A; ANOVA; F_{15,32}=189.939, p<0.0005). The supernatant of a full Lb. fermentum APC249 preparation did not stimulate Bifidobacterium growth (Bonferroni post-hoc, compared to water p=1), while its half concentration did stimulate the growth (Bonferroni post-hoc, compared to water p<0.0005), albeit not to the level of full (Bonferroni post-hoc compared to LLL p<0.0005, compared to Lb. fermentum 1b p=0.024) and a half strength supernatants of Lb. fermentum 1b and LLL (Bonferroni post-hoc compared to ½ LLL Sup. p=0.009, compared to ½Lb. fermentum 1b Sup. p<0.0005). Concentration dependent effects on 24 h growth of Bifidobacterium were also observed when using preparations containing both supernatant and cells (FIG. 9B; ANOVA; F_{12,26}=120.838, p<0.0005). Full concentrations of Lb. delbrueckii APC2421, Lb. delbrueckii APC2516, and Lb. reuteri APC2482 were not able to stimulate 24 h growth of Bifidobacterium (Bonferroni post-hoc, compared to water p=1, p=0.912, and p=1, respectively), however their half concentrations did stimulate growth (Bonferroni post-hoc, compared to water all p<0.0005), albeit not to the levels observed for LLL (Bonferroni post-hoc, compared to LLL all p<0.0005). In contrast, the half concentration of LLL caused less stimulation compared to full LLL (Bonferroni post-hoc, p=0.003), while half and full concentrations of Lb. delbrueckii 2z (Bonferroni post-hoc, p=0.104) and Lb. delbrueckii ssp. bulgaricus APC2493 (Bonferroni post-hoc, p=0.112) showed comparable stimulation independent of concentration.

### Effect of MRS on Bifidobacteria

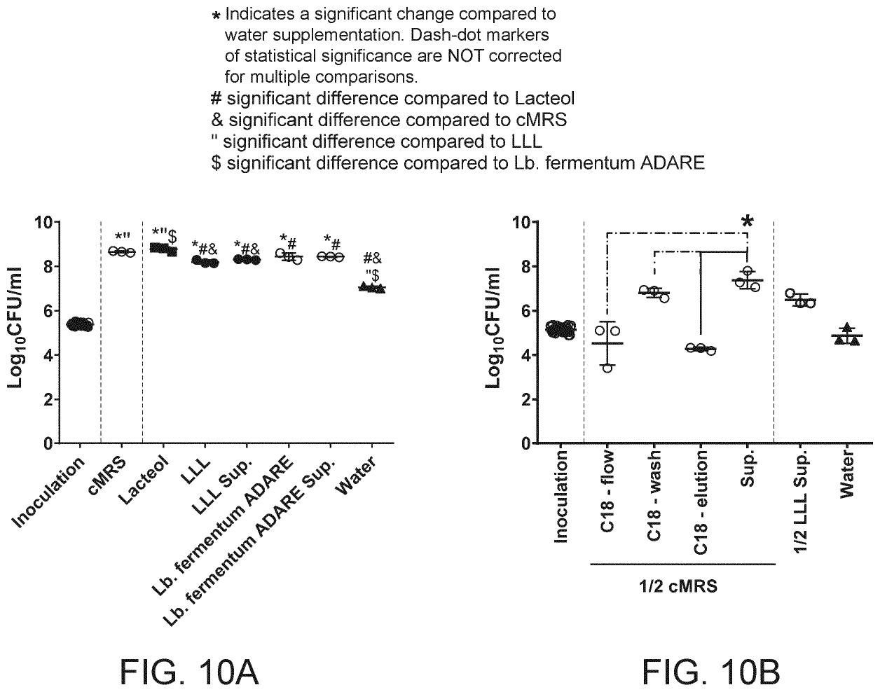

In 10× diluted media, concentrated MRS (cMRS; weight equivalent of LACTEOL®) stimulated Bifidobacterium growth to a degree comparable to LACTEOL® (FIG. 10A; ANOVA; F_{3,8}=12.078, p=0.002; Bonferroni post-hoc, p=1) and better than LLL and its supernatant (Bonferroni post-hoc, p<0.003). There were differences in Bifidobacterium counts after supplementation with C18 fractions of half concentrated MRS (½ cMRS) (FIG. 10B; Independent samples Kruskal-Wallis; Kruskal-Wallis, χ² (5)=15.222, p=0.009). ½ cMRS, followed by ½ cMRS C18 wash fraction, having numerically highest counts.

### Conclusions

In Example 1 it was demonstrated that LACTEOL® supplementation increased both relative and absolute abundance of bifidobacteria during a 24 h human fecal fermentation. Example 2 confirms the fermenter data in that LACTEOL® stimulates the growth of bifidobacteria. This is illustrated, for example, by growth stimulation of 71% (five out of seven) of tested Bifidobacterium strains isolated from both infants and adult individuals. LACTEOL® activity was dose-dependent with the highest activity seen at 34 mg/ml, while a 100 times lower dose (0.34 mg/ml) has no effect.

Prebiotics are commonly used to stimulate bifidobacteria growth, in particular lactose related compounds, such as lactulose and GOS, as well as inulin and FOS are known for stimulation of bifidobacteria growth. However, bifidobacteria stimulation was not attributed to the lactose present in LACTEOL® as the low lactose version of LACTEOL® (LLL) showed comparable activity to LACTEOL®.

The ability to stimulate Bifidobacterium growth is not a common trait among probiotic preparations. Three commercially available bacterial products at their daily doses did not stimulate Bifidobacterium growth. These included spores of B. clausii, cells of Lb. reuteri DSM 17938 and Lb. rhamnosus GG. Interestingly, supplementation with the Lb. rhamnosus GG preparation contained inulin, yet despite the presence of this well-known prebiotic, completely inactivated Bifidobacterium cells. In contrast, a yeast-based preparation containing S. boulardii CNCM 1-745 stimulated Bifidobacterium growth in a manner similar to LACTEOL®.

Both LACTEOL® supernatant and cell fractions showed comparable stimulation of bifidobacteria growth. However, LLL cells and Lb. fermentum 1b cells did not show equal stimulation suggesting that lactose addition and/or spray drying/lyophilization process have an impact on the activity of the cell fractions.

Among the two strains used in LACTEOL® production, only Lb. fermentum showed stimulation of Bifidobacterium growth comparable to LACTEOL®, while Lb. delbrueckii showed no or minimal growth enhancement. Furthermore, none of the five other full-strength preparations generated by the tested lactobacilli was able to stimulate bifidobacteria growth to the LACTEOL® level. Unexpectedly, half concentrations of those preparations had a more diverse impact on bifidobacteria growth. Four of the half-strength preparations showed a stimulatory effect (Lb. fermentum APC 249, Lb. delbrueckii APC 2421, Lb. delbrueckii APC 2516, Lb. reuterii APC 2482), once again not to the level of LACTEOL®. This is another unexpected result, in that full-strength preparations of these four strains are inhibitory, whereas half-strength preparations have a stimulatory effect.

Several separation experiments were conducted to identify one or more compounds responsible for bifidobacteria growth stimulation. Firstly, it was demonstrated that compounds responsible for LACTEOL® activity could be partially purified using C18 columns, normally used for extraction of hydrophobic or polar organic analytes from aqueous matrices. Active compounds were liberated from the column with the 5% methanol wash, indicating that those compounds were only weakly bound to the column sorbent. In general, reconstituted wash fractions of half strength preparations of LACTEOL®, LLL, and Lb. fermentum 1b were as active as their non-purified preparations, yet they had much cleaner chromatograms. When wash fractions were concentrated to full concentrations their activity was equal to the LACTEOL® supernatant, once again demonstrating that inhibitory compounds were somewhat removed. Interestingly, C18 purification of Lb. fermentum APC 249 removed not only impurities but also the bifidostatic compounds produced by this strain. Secondly, concentrated fractions collected from a size exclusion column were tested in the bifidogenic assay revealing that Fraction 52 has the potential to encompass the growth stimulatory compounds, while MALDI TOF mass spectrometry of Fraction 52 resulted in a potentially interesting peak.

1. A composition comprising cells of Lactobacillus fermentum for use in maintaining and/or improving gut health of a human or non-human animal subject by stimulating the growth of bifidobacteria in the human or non-human animal gut.

2. The composition for use according to claim 1, also comprising cells of Lactobacillus delbrueckii.

3. A composition comprising the culture medium in which cells of Lactobacillus fermentum have been grown for use in maintaining and/or improving gut health of a human or non-human animal subject by stimulating the growth of bifidobacteria in the human or non-human animal gut.

4. The composition for use according to claim 1, also comprising the culture medium in which cells of Lactobacillus fermentum have been grown.

5. The composition for use according to claim 2, also comprising the culture medium in which cells of Lactobacillus fermentum and cells of Lactobacillus delbrueckii have been grown.

6. A composition comprising the supernatant of the culture medium in which cells of Lactobacillus fermentum and cells of Lactobacillus delbrueckii have been grown for use in maintaining and/or improving gut health of a human or non-human animal subject by stimulating the growth of bifidobacteria in the human or non-human animal gut.

7. The composition for use according to claim 6, comprising Fraction 52 of the supernatant, wherein Fraction 52 is a size exclusion HPLC fraction with a single peak at around 5200 m/z in MALDI TOF mass spectrometry.

8. The composition for use according to claim 2, wherein the cells of Lactobacillus fermentum and/or Lactobacillus delbrueckii are dead cells.

9. The composition for use according to claim 8, comprising LACTEOL®.

10. The composition for use according to claim 1, wherein the subject is a healthy human.

11. The composition for use according to claim 1, wherein the subject is a human with a disorder of the gut.

12. The composition for use according to claim 11, wherein the gut disorder is selected from antibiotic-associated diarrhea, dysbiosis, irritable bowel syndrome (IBS) and inflammatory bowel disease (IBD).

13. The composition for use according to claim 1, wherein the composition is in the form of a pharmaceutical composition, a food supplement, or a nutritional supplement.

14. The composition for use according to claim 13, wherein said food supplement or nutritional supplement is comprised within a food product selected from milk, yoghurt or yoghurt-style product, cheese, ice-cream, a cereal-based product, a milk-based powder, an infant formula, a nutritional formula, a dried oral grit or powder, a wet oral paste or jelly, a grit or powder for dry tube feeding or a fluid for wet tube feeding.

15. The composition for use according to claim 9, wherein the subject is a healthy human.

16. The composition for use according to claim 9, wherein the subject is a human with a disorder of the gut.

17. The composition for use according to claim 16, wherein the gut disorder is selected from antibiotic-associated diarrhea, dysbiosis, irritable bowel syndrome (IBS) and inflammatory bowel disease (IBD).

18. The composition for use according to claim 9, wherein the composition is in the form of a pharmaceutical composition, a food supplement, or a nutritional supplement.

19. The composition for use according to claim 18, wherein said food supplement or nutritional supplement is comprised within a food product selected from milk, yoghurt or yoghurt-style product, cheese, ice-cream, a cereal-based product, a milk-based powder, an infant formula, a nutritional formula, a dried oral grit or powder, a wet oral paste or jelly, a grit or powder for dry tube feeding or a fluid for wet tube feeding.


SEQUENCE LISTING (6 sequences)

SEQ ID NO: 1 - DNA, 50 bp, Artificial Sequence
  synthetic construct
  misc_feature (42)..(42): n is a, c, g, or t
tcgtcggcagcgtcagatgtgtataagagacagcctacgggnggcwgcag

SEQ ID NO: 2 - DNA, 55 bp, Artificial Sequence
  synthetic construct
gtctcgtgggctcggagatgtgtataagagacaggactachvgggtatctaatcc

SEQ ID NO: 3 - DNA, 21 bp, Artificial Sequence
  synthetic construct
actcctacgggaggcagcagt

SEQ ID NO: 4 - DNA, 19 bp, Artificial Sequence
  synthetic construct
tattaccgcggctgctggc

SEQ ID NO: 5 - DNA, 18 bp, Artificial Sequence
  synthetic construct
cgtccgttctacccgatg

SEQ ID NO: 6 - DNA, 18 bp, Artificial Sequence
  synthetic construct
ggtcttcttgccgtcgat